(19) United States
(12) Patent Application Publication
(10) Pub. No.: US 2016/0068901 A1
(43) Pub. Date: Mar. 10, 2016

(54) Analysis of DNA

(71) Applicant: ADVANCED LIQUID LOGIC, INC., San Diego, CA (US)

(72) Inventors: Allen E. Eckhardt, San Diego, CA (US); Michael G. Pollack, Durham, NC (US); David S. Cohen, Chapel Hill, NC (US)

(73) Assignee: Advanced Liquid Logic, Inc., San Diego, CA (US)

(21) Appl. No.: 14/888,137

(22) PCT Filed: May 1, 2014
(86) PCT No.: PCT/US14/36407

§ 371 Date: Oct. 30, 2015

Related U.S. Application Data
(60) Provisional application No. 61/924,010, filed on Jan. 6, 2014.
(60) Provisional application No. 61/870,357, filed on Aug. 27, 2013.
(60) Provisional application No. 61/834,039, filed on Jun. 12, 2013.
(60) Provisional application No. 61/828,232, filed on May 29, 2013.
(60) Provisional application No. 61/817,925, filed on May 1, 2013.

(51) Int. Cl. C12Q 1/68

(52) U.S. Cl. CPC C12Q 1/6869

(57) ABSTRACT

The invention provides pyrosequencing-based methods of analyzing and synthesizing DNA, including methods of DNA error correction, determining DNA size distribution, screening for nucleotide repeat disorders such as fragile X syndrome, determining size distribution and bias in a DNA library, and determining pyrosequencing read length. The methods include on-bench protocols as well as droplet-based protocols that may be conducted on a droplet actuator.

### RELATED APPLICATIONS

In addition to the patent applications cited herein, each of which is incorporated herein by reference, this patent application is related to and claims priority to U.S. Provisional Patent Application No. 61/817,925, filed on May 1, 2013, entitled “Analysis of DNA;” U.S. Provisional Patent Application No. 61/828,232, filed on May 29, 2013, entitled “Analysis of DNA;” U.S. Provisional Patent Application No. 61/834,039, filed on Jun. 12, 2013, entitled “Analysis of DNA;” U.S. Provisional Patent Application No. 61/870,357, filed on Aug. 27, 2013, entitled “Analysis of DNA;” and U.S. Provisional Patent Application No. 61/924,010, filed on Jan. 6, 2014, entitled “Analysis of DNA;” the entire disclosures of which are incorporated herein by reference.

### FIELD OF THE INVENTION

The present invention generally relates to the field of pyrosequencing-based methods of analyzing DNA. In particular, the present invention provides a method of DNA error correction comprising the use of pyrosequencing chemistry.

### BACKGROUND

A droplet actuator typically includes one or more substrates configured to form a surface or gap for conducting droplet operations. The one or more substrates establish a droplet operations surface or gap for conducting droplet operations and may also include electrodes arranged to conduct the droplet operations. The droplet operations substrate or the gap between the substrates may be coated or filled with a filler fluid that is immiscible with the liquid that forms the droplets.

Droplet actuators are used to conduct a variety of molecular techniques that are commonly used to analyze a DNA sample. The DNA sample may, for example, be a mixture of sequences, such as a mixture of synthesized strands or fragments in a nucleic acid library, or a clinical DNA sample used in a diagnostic or screening assay. In one example, analysis of the DNA sample may include determining the size distribution of DNA fragments in the sample. In another example, analysis of the DNA sample may include ensuring the accuracy of the DNA sequence in the sample. DNA fragment size distribution and DNA sequence accuracy are typically determined using different molecular techniques that often require different reagents and equipment. There is a need for new approaches for analyzing a DNA sample that is based on a single molecular technique.

### BRIEF DESCRIPTION

A pyrosequencing method for DNA error correction is provided comprising: a) synthesizing DNA molecules comprising a nucleotide sequence of a template DNA molecule to produce a DNA sample; b) performing a DNA error correction method, the method comprising pyrocorrection to reduce or eliminate imperfect DNA strands in the DNA sample; and c) amplifying the DNA molecules in the DNA sample to increase the quantity of perfect DNA strands in the DNA sample. In one embodiment, pyrocorrection may comprise: i) blocking the synthesis of a DNA molecule when the next base to be added during primer extension differs from an expected base as compared to the nucleotide sequence of the template DNA molecule; ii) adding the expected base to the DNA molecule as compared to the nucleotide sequence of the template DNA molecule; and iii) repeating steps (i) and (ii) until the synthesis of a DNA molecule comprising the nucleotide sequence of a template DNA molecule is complete, and wherein synthesis of a DNA molecule that does not comprise the expected nucleotide sequence as compared to the nucleotide sequence of a template DNA molecule is blocked. In another embodiment, pyrocorrection may comprise: i) coupling DNA molecules in the DNA sample to beads; ii) denaturing the DNA molecules; iii) washing the beads to yield single stranded DNA molecules coupled to the beads; iv) annealing primers to the single stranded DNA molecules coupled to the beads; v) blocking the synthesis of a DNA molecule when the next base to be added during primer extension differs from an expected base as compared to the nucleotide sequence of a template DNA molecule; vi) washing the beads; vii) adding the expected base to the DNA molecule as compared to the nucleotide sequence of the template DNA molecule; viii) washing the beads; and ix) repeating some or all of steps (v) to (viii) until the synthesis of a DNA molecule comprising the nucleotide sequence of the template DNA molecule is complete.

In some embodiments of the pyrosequencing DNA error correction methods of the invention, blocking the synthesis of a DNA molecule comprises adding complementary blocking bases and reagents for adding the complementary blocking bases during primer extension of the DNA molecule being synthesized, wherein the complementary blocking bases comprise each of the three bases that are not the expected base as compared to the nucleotide sequence of a template DNA molecule. In other embodiments, adding the expected base to the DNA molecule as compared to the nucleotide sequence of the template DNA molecule comprises adding bases and reagents for adding the bases during primer extension of the DNA molecule being synthesized, wherein the bases comprise the expected base as compared to the nucleotide sequence of the template DNA molecule. In further embodiments, the number of perfect DNA strands in the DNA sample is increased by at least 1.5×, at least 2×, at least 3×, at least 4×, or at least 5×.

In other embodiments, the pyrosequencing DNA error correction methods of the invention are combined with one or more additional DNA error correction methods, such as an enzyme-surveillance error correction method. The methods may also comprise high fidelity DNA synthesis conditions using high fidelity DNA polymerases. In such embodiments, the number of perfect DNA strands in the DNA sample may be increased to at least 90%, at least 91%, at least 92%, at least 93%, at least 94%, at least 95%, at least 96%, at least 97%, at least 98%, or at least 99% of the DNA molecules in the DNA sample. In other embodiments, the nucleotide sequence of the template DNA molecule may be from 100 to 1,000 base pairs, or may be from 1,000 to 10,000 base pairs.

In some embodiments, the pyrocorrection methods of the invention further comprise incorporating nucleotides in a region of the DNA molecule comprising homopolymeric runs. In further embodiments, the pyrocorrection methods further comprise synthesis of DNA molecules comprising dinucleotides, trinucleotides, and/or other polynucleotides.

A method of determining the average size of DNA fragments in a DNA sample is also provided, the method comprising: a) conducting a pyrosequencing reaction comprising combining the DNA sample and pyrosequencing reagents, wherein the pyrosequencing reaction is conducted without determining the nucleic acid sequences of the DNA fragments in the DNA sample, whereby the pyrosequencing reaction yields a detectable pyrophosphate concentration; b) determining the pyrophosphate concentration; and c) determining the average size of DNA fragments in the DNA sample based on the pyrophosphate concentration. Combining the DNA sample and pyrosequencing reagents may include incubating the DNA sample with terminal deoxytransferase and ddATP, wherein dideoxynucleotides are incorporated into the DNA fragments in the DNA sample. The pyrophosphate concentration in the DNA sample may be determined in moles/liter, and particularly may be determined by performing a chemiluminescence assay on the DNA sample. In addition, determining the average size of DNA fragments in the DNA sample based on the pyrophosphate concentration may comprise the steps of: i) determining a DNA concentration in the DNA sample in grams/liter; ii) calculating the average molecular weight of the DNA fragments in the DNA sample in grams/mole, comprising dividing the DNA concentration in grams/liter by ½ the pyrophosphate concentration in moles/liter; and iii) calculating the average size of DNA fragments in the DNA sample, comprising dividing the average molecular weight of DNA in grams/mole by 660 grams/base pair. In other embodiments, determining a DNA concentration in the DNA sample comprises performing qPCR on the DNA sample.

A method for diagnosing or screening for CGG trinucleotide repeats in a Fragile X-mental retardation (FMR1) gene in a biological sample comprising genomic DNA is also provided, the method comprising: a) purifying the genomic DNA from the biological sample; b) amplifying a CGG trinucleotide repeat domain in the genomic DNA by PCR cycling, thereby producing amplified DNA nucleotide sequences; c) preparing DNA templates from the amplified DNA nucleotide sequences; and d) pyrosequencing the DNA templates. In some embodiments, pyrosequencing the DNA templates comprises a nucleotide natural block method comprising alternating the presentation of dCTP or dGTP nucleotides until the blocking nucleotides are reached, thereby identifying the 3′ end of the CGG trinucleotide repeat domain.

In other embodiments, a method of screening for CGG trinucleotide repeats in a FMR1 gene in a biological sample comprising genomic DNA on a droplet actuator is provided, the method comprising: a) transferring the biological sample comprising genomic DNA to a sample preparation reservoir of the droplet actuator; b) purifying the genomic DNA from the biological sample in the sample preparation reservoir of the droplet actuator, thereby producing an eluted DNA droplet; c) amplifying a CGG trinucleotide repeat domain in the eluted DNA droplet by PCR cycling, thereby producing amplified DNA nucleotide sequences; d) preparing single stranded DNA (ssDNA) templates from the amplified DNA nucleotide sequences; e) pyrosequencing the ssDNA templates on the droplet actuator; and f) detecting a luminescent signal from the ssDNA templates, whereby the number of CGG trinucleotide repeats in the FMR1 gene in the biological sample is enumerated. Enumeration of CGG trinucleotide repeats may be used to diagnose Fragile X syndrome in a newborn or determine whether an individual is a carrier of the FRM1 mutation.

A method of determining the size distribution and bias in a DNA library is also provided, the method comprising: a) providing a DNA sample; b) pyrosequencing DNA molecules in the DNA samples, thereby producing pyrosequencing data; c) fitting a curve to the pyrosequencing data; and d) characterizing library size distribution and bias based on characteristics of the curve. In some embodiments, pyrosequencing DNA molecules in the DNA samples comprises: i) incorporating a first mixture of dATP and dTTP; ii) incorporating a second mixture of dGTP and dCTP; and iii) repeating steps (i) and (ii) until complementary strands in the DNA sample are completely synthesized. Fitting the curve to the pyrosequencing data may comprise a nonlinear fit of the pyrosequencing data to generate output data comprising GC-content, standard deviation of AT-content, standard deviation of GC-content, average fragment size, and/or standard deviation of fragment size.

A method of determining pyrosequencing read length using dATP and apyrase in a pyrosequencing reaction on a droplet actuator is also provided, the method comprising: a) combining a DNA template droplet with a reagent droplet to yield a reaction droplet, wherein the reagent droplet comprises dATP and Klenow DNA polymerase, whereby dATP is incorporated into the DNA template in the reaction droplet; b) combining the reaction droplet with a droplet comprising apyrase to yield a reaction/apyrase droplet, wherein the apyrase degrades unincorporated dATP; c) combining the reaction/apyrase droplet with a droplet comprising an apyrase inhibitor to yield a dATP-free droplet; d) combining the dATP-free droplet with a droplet comprising sulfurylase and luciferase, whereby a detectable luminescent signal is produced; and e) detecting the luminescent signal, whereby the luminescent signal is indicative of pyrosequencing read length.

A method for synthesizing a DNA molecule on a droplet actuator is also provided, the method comprising: a) providing a sample comprising a set of oligonucleotides designed for a region of a DNA molecule of interest such that the ends of each oligonucleotide overlap other oligonucleotides in the set of oligonucleotides; b) transferring the sample to a sample input reservoir of the droplet actuator; c) dispensing an oligonucleotide droplet from the sample input reservoir and combining the oligonucleotide droplet with an assembly reagent droplet to yield an assembly droplet; d) transporting the assembly droplet to a temperature control zone on the droplet actuator; e) incubating the assembly droplet, whereby DNA cassettes are assembled in the assembly droplet; f) amplifying the assembled DNA cassettes using PCR cycling; g) performing DNA error correction on the DNA cassettes to yield error-corrected DNA cassettes; h) amplifying the error-corrected DNA cassettes using PCR cycling; and i) collecting the error-corrected DNA cassettes. In some embodiments, the DNA error correction may comprise a pyrocorrection method and/or an enzyme-surveillance error correction method.

On-bench protocols as well as droplet-based protocols that may be conducted on a droplet actuator are provided, as well as microfluidics systems programmed to execute the method of any of the methods of the invention on a droplet actuator.

### DEFINITIONS

As used herein, the following terms have the meanings indicated.

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 1000 V, or about 300 V. Where alternating current is used, any suitable frequency may be employed. For example, an electrode may be activated using alternating current having a frequency from about 1 Hz to about 10 MHz, or from about 10 Hz to about 60 Hz, or from about 20 Hz to about 40 Hz, or about 30 Hz.

“Amplify,” “amplification,” “nucleic acid amplification,” or the like, refers to the production of multiple copies of a nucleic acid template (e.g., a template DNA molecule), or the production of multiple nucleic acid sequence copies that are complementary to the nucleic acid template (e.g., a template DNA molecule).

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a flow path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids. A droplet may include one or more beads.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000; Kim and/or Shah et al., U.S. patent application Ser. No. 10/343,261, entitled “Electrowetting-driven Micropumping,” filed on Jan. 27, 2003, Ser. No. 11/275,668, entitled “Method and Apparatus for Promoting the Complete Transfer of Liquid Drops from a Nozzle,” filed on Jan. 23, 2006, Ser. No. 11/460,188, entitled “Small Object Moving on Printed Circuit Board,” filed on Jan. 23, 2006, Ser. No. 12/465,935, entitled “Method for Using Magnetic Particles in Droplet Microfluidics,” filed on May 14, 2009, and Ser. No. 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. No. 7,641,779, entitled “Method and Apparatus for Programmable fluidic Processing,” issued on Jan. 5, 2010, and U.S. Pat. No. 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, U.S. Patent Pub. No. 20110048951, entitled “Digital Microfluidics Based Apparatus for Heat-exchanging Chemical Processes,” published on Mar. 3, 2011; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a droplet operations gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact or frequent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a flow path from the reservoir into the droplet operations gap). Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), other fluorinated monomers for plasma-enhanced chemical vapor deposition (PECVD), and organosiloxane (e.g., SiOC) for PECVD. In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass), PARYLENE™ N, and PARYLENE™ HT (for high temperature, ˜300° C.) (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; polypropylene; and black flexible circuit materials, such as DuPont™ Pyralux® HXC and DuPont™ Kapton® MBC (available from DuPont, Wilmington, Del.). Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, other fluorinated monomers for plasma-enhanced chemical vapor deposition (PECVD), and organosiloxane (e.g., SiOC) for PECVD. Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing or imaging techniques may sometimes be used to determine or confirm the outcome of a droplet operation. Examples of such techniques are described in Sturmer et al., U.S. Patent Application Publication No. US20100194408, entitled “Capacitance Detection in a Droplet Actuator,” published on Aug. 5, 2010, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1×-, 2×-3×-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be or include a low-viscosity oil, such as silicone oil or hexadecane filler fluid. The filler fluid may be or include a halogenated oil, such as a fluorinated or perfluorinated oil. The filler fluid may fill the entire gap of the droplet actuator or may coat one or more surfaces of the droplet actuator. Filler fluids may be conductive or non-conductive. Filler fluids may be selected to improve droplet operations and/or reduce loss of reagent or target substances from droplets, improve formation of microdroplets, reduce cross contamination between droplets, reduce contamination of droplet actuator surfaces, reduce degradation of droplet actuator materials, etc. For example, filler fluids may be selected for compatibility with droplet actuator materials. As an example, fluorinated filler fluids may be usefully employed with fluorinated surface coatings. Fluorinated filler fluids are useful to reduce loss of lipophilic compounds, such as umbelliferone substrates like 6-hexadecanoylamido-4-methylumbelliferone substrates (e.g., for use in Krabbe, Niemann-Pick, or other assays); other umbelliferone substrates are described in U.S. Patent Pub. No. 20110118132, published on May 19, 2011, the entire disclosure of which is incorporated herein by reference. Examples of suitable fluorinated oils include those in the Galden line, such as Galden HT170 (bp=170° C., viscosity=1.8 cSt, density=1.77), Galden HT200 (bp=200 C, viscosity=2.4 cSt, d=1.79), Galden HT230 (bp=230 C, viscosity=4.4 cSt, d=1.82) (all from Solvay Solexis); those in the Novec line, such as Novec 7500 (bp=128 C, viscosity=0.8 cSt, d=1.61), Fluorinert FC-40 (bp=155° C., viscosity=1.8 cSt, d=1.85), Fluorinert FC-43 (bp=174° C., viscosity=2.5 cSt, d=1.86) (both from 3M). In general, selection of perfluorinated filler fluids is based on kinematic viscosity (<7 cSt is preferred, but not required), and on boiling point (>150° C. is preferred, but not required, for use in DNA/RNA-based applications (PCR, etc.)). Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein. Fluorinated oils may in some cases be doped with fluorinated surfactants, e.g., Zonyl FSO-100 (Sigma-Aldrich) and/or others.

“Immobilize” with respect to magnetically responsive beads, means that the beads are substantially restrained in position in a droplet or in filler fluid on a droplet actuator. For example, in one embodiment, immobilized beads are sufficiently restrained in position in a droplet to permit execution of a droplet splitting operation, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe12O19, CoO, NiO, Mn2O3, Cr2O3, and CoMnP.

“Nucleic acid” as used herein means a polymeric compound comprising covalently linked subunits called nucleotides. A “nucleotide” is a molecule, or individual unit in a larger nucleic acid molecule, comprising a nucleoside (i.e., a compound comprising a purine or pyrimidine base linked to a sugar, usually ribose or deoxyribose) linked to a phosphate group.

“Polynucleotide” or “oligonucleotide” or “nucleic acid molecule” are used interchangeably herein to mean the phosphate ester polymeric form of ribonucleosides (adenosine, guanosine, uridine or cytidine; “RNA molecules” or simply “RNA”) or deoxyribonucleosides (deoxyadenosine, deoxyguanosine, deoxythymidine, or deoxycytidine; “DNA molecules” or simply “DNA”), or any phosphoester analogs thereof, such as phosphorothioates and thioesters, in either single-stranded or double-stranded form. Polynucleotides comprising RNA, DNA, or RNA/DNA hybrid sequences of any length are possible. Polynucleotides for use in the present invention may be naturally-occurring, synthetic, recombinant, generated ex vivo, or a combination thereof, and may also be purified utilizing any purification methods known in the art. Accordingly, the term “DNA” includes but is not limited to genomic DNA, plasmid DNA, synthetic DNA, semi-synthetic DNA, complementary DNA (“cDNA”; DNA synthesized from a messenger RNA template), and recombinant DNA (DNA that has been artificially designed and therefore has undergone a molecular biological manipulation from its natural nucleotide sequence). A “gene” as used herein, refers to a polynucleotide containing at least one open reading frame that is capable of encoding a particular protein after being transcribed and translated.

“Polynucleotide fragment” as used herein means a polynucleotide of reduced length relative to a reference polynucleotide and comprising, over the common portion, a nucleotide sequence identical to that of the reference polynucleotide. Such a polynucleotide fragment may be, where appropriate, included in a larger polynucleotide of which it is a constituent. Such polynucleotide fragments comprise, or alternatively consist of, polynucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000 or 1500 consecutive nucleotides of a reference polynucleotide. Polynucleotide fragments include, for example, DNA fragments and RNA fragments.

“Protocol” means a series of steps that includes, but is not limited to, droplet operations on one or more droplet microactuators and/or DNA synthesis or sequencing.

“Reservoir” means an enclosure or partial enclosure configured for holding, storing, or supplying liquid. A droplet actuator system of the invention may include on-cartridge reservoirs and/or off-cartridge reservoirs. On-cartridge reservoirs may be (1) on-actuator reservoirs, which are reservoirs in the droplet operations gap or on the droplet operations surface; (2) off-actuator reservoirs, which are reservoirs on the droplet actuator cartridge, but outside the droplet operations gap, and not in contact with the droplet operations surface; or (3) hybrid reservoirs which have on-actuator regions and off-actuator regions. An example of an off-actuator reservoir is a reservoir in the top substrate. An off-actuator reservoir is typically in fluid communication with an opening or flow path arranged for flowing liquid from the off-actuator reservoir into the droplet operations gap, such as into an on-actuator reservoir. An off-cartridge reservoir may be a reservoir that is not part of the droplet actuator cartridge at all, but which flows liquid to some portion of the droplet actuator cartridge. For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap. A system using an off-cartridge reservoir will typically include a fluid passage means whereby liquid may be transferred from the off-cartridge reservoir into an on-cartridge reservoir or into a droplet operations gap.

“Sequence identity” or “identity” in the context of nucleic acid sequences and as known in the art refers to the nucleic acid bases in two sequences that are the same when aligned for maximum correspondence over a specified comparison window. Thus, “percent sequence identity” refers to the value determined by comparing two optimally aligned sequences over a comparison window, wherein the portion of the polynucleotide sequence in the comparison window may comprise additions or deletions (i.e., gaps) as compared to the reference or template sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the results by 100 to yield the percentage of sequence identity. Useful examples of percent sequence identities include, but are not limited to any integer percentage from 50% to 100%, in particular 50%, 55%, 60%, 65%, 70%, 75%, 80%, 85%, 90%, 91%, 92%, 93%, 94%, 95%, 96%, 97%, 98%, 99%, or 100%. Sequence alignments and percent sequence identity calculations may be performed using methods and sequence analysis software known in the art, including but not limited to, the MegAlign™ program of the LASERGENE bioinformatics computing suite (DNASTAR Inc., Madison, Wis.), multiple alignment using the Clustal method (Higgins and Sharp (1989) CABIOS. 5:151-153) with the default parameters, including default parameters for pairwise alignments, the GCG suite of programs (Wisconsin Package Version 9.0, Genetics Computer Group (GCG), Madison, Wis.), BLASTP, BLASTN, BLASTX (Altschul et al. (1990) J. Mol. Biol. 215:403-410, and DNASTAR (DNASTAR, Inc., Madison, Wis.). Within the context of this application it will be understood that where sequence analysis software is used for analysis, the results of the analysis will be based on the default values of the program referenced, unless otherwise specified (i.e., any set of values or parameters which originally load with the software when first initialized).

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in any of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated away from a certain region of the magnetic field, in each case where the magnetic field in such region is not capable of substantially attracting any magnetically responsive beads in the droplet or in which any remaining attraction does not eliminate the effectiveness of droplet operations conducted in the region. In various aspects of the invention, a system, a droplet actuator, or another component of a system may include a magnet, such as one or more permanent magnets (e.g., a single cylindrical or bar magnet or an array of such magnets, such as a Halbach array) or an electromagnet or array of electromagnets, to form a magnetic field for interacting with magnetically responsive beads or other components on chip. Such interactions may, for example, include substantially immobilizing or restraining movement or flow of magnetically responsive beads during storage or in a droplet during a droplet operation or pulling magnetically responsive beads out of a droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface. In one example, filler fluid can be considered as a film between such liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### BRIEF DESCRIPTION OF THE DRAWINGS

FIG. 1 illustrates a flow diagram of an example of a method of DNA error correction;

FIG. 2 illustrates a flow diagram of an example of a method of pyrocorrection;

FIG. 3 illustrates a flow diagram of an example of a method that describes in more detail the method of FIG. 1;

FIG. 4 illustrates a flow diagram of an example of a method of DNA error correction that includes enzyme-surveillance and pyrocorrection;

FIG. 5 illustrates a flow diagram of an example of a method of calculating the average size of DNA fragments;

FIG. 6 illustrates a flow diagram of an example of a method for diagnosing or screening for nucleotide repeat disorders;

FIG. 7 illustrates a flow diagram of a method of determining size distribution and bias in a library of nucleic acids;

FIGS. 8A and 8B show a pyrogram and a plot, respectively, of pyrosequencing results and analysis of a simulated non-biased nucleic acid library;

FIGS. 9A and 9B show a pyrogram and a plot, respectively, of pyrosequencing results and analysis of a simulated biased nucleic acid library;

FIG. 10 illustrates an example of a method of using dATP and apyrase in a pyrosequencing assay performed on a droplet actuator;

FIG. 11 illustrates a flow diagram of an example of a protocol for synthesis of a DNA molecule on a droplet actuator; and

FIG. 12 illustrates a functional block diagram of an example of a microfluidics system that includes a droplet actuator.

### DESCRIPTION

The invention provides pyrosequencing-based methods of analyzing DNA. In one embodiment, the invention provides a method of DNA error correction comprising the use of pyrosequencing chemistry. In one embodiment, the method of DNA error correction comprises increasing the number of perfect DNA strands in a DNA sample, for example wherein the DNA sample comprises synthesized DNA strands.

In another embodiment, the invention provides a method of determining the average size of DNA fragments in a DNA sample. In one embodiment, the method of determining the average size of DNA fragments in a DNA sample comprises assessing the quality of a DNA sample (e.g., a bio-banked DNA sample). In another embodiment, the method of determining the average size of DNA fragments in a DNA sample comprises a method for diagnosing or screening for nucleotide repeat disorders.

In yet another embodiment, the invention provides a method of characterizing a library of nucleic acids comprising determining the quality of a nucleic acid library that may be used in a next-generation sequencing protocol. In one embodiment, the method of the invention may be used to determine the guanine-cytosine content (GC-content) of a nucleic acid library. In a further embodiment, GC-content is used as an indicator that the nucleic acid library comprises the appropriate DNA. In another embodiment, standard deviations of AT-content and GC-content are used as an indicator of bias in the nucleic acid library, wherein nucleic acid library bias is calculated to show that no single DNA molecule or set of DNA molecules are over-represented in the nucleic acid library. In another embodiment, the method of the invention may be used to determine the average size of DNA fragments in the nucleic acid library and the standard deviation of DNA fragment size, wherein the average size of DNA fragments in the nucleic acid library is calculated to determine whether the DNA fragments in the nucleic acid library are of suitable size and wherein the standard deviation of DNA fragment size is calculated to determine whether the distribution of DNA fragments in the nucleic acid library is within a suitable range.

### 7.1 Error Correction

The invention provides a new method of DNA error correction, comprising the use of pyrosequencing chemistry. Accordingly, the DNA error correction method of the present invention comprises a “pyrocorrection” method. In one embodiment, the method of DNA error correction comprises synthesizing DNA molecules comprising the nucleotide sequence of a template DNA molecule to produce a DNA sample; using a DNA error correction method, the method comprising pyrocorrection to reduce or eliminate imperfect DNA strands in the DNA sample; and amplifying the DNA in the DNA sample to increase the quantity of perfect DNA strands in the DNA sample. “Imperfect DNA strand” as used herein means a DNA molecule comprising a nucleotide sequence having less than 100% sequence identity to the nucleotide sequence of a template DNA molecule. “Perfect DNA strand” as used herein means a DNA molecule comprising a nucleotide sequence having 100% sequence identity to the nucleotide sequence of a template DNA molecule.

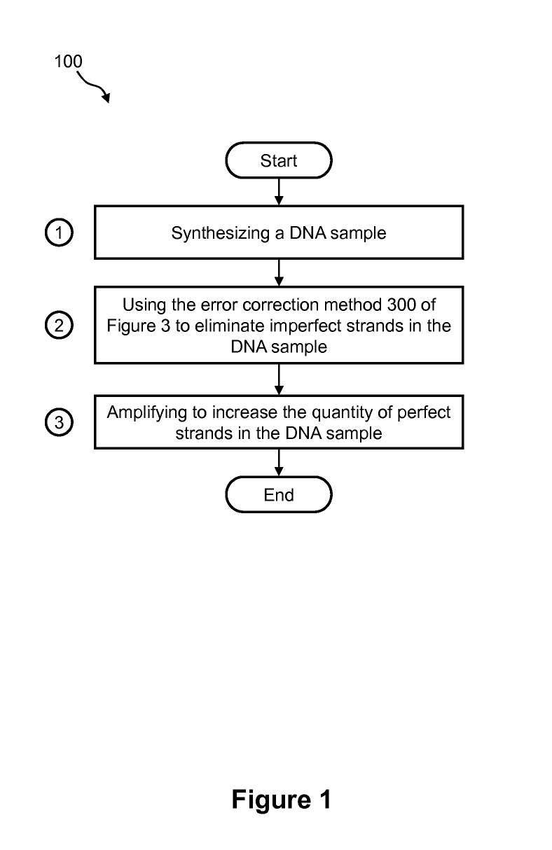

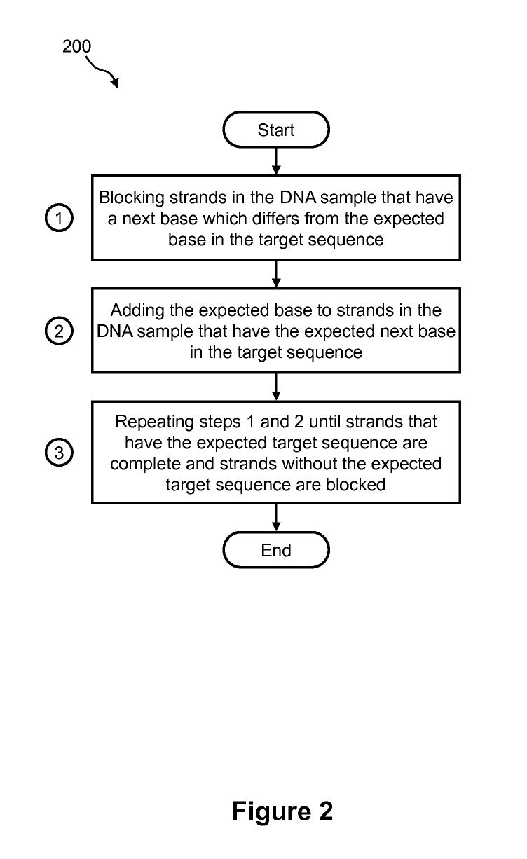

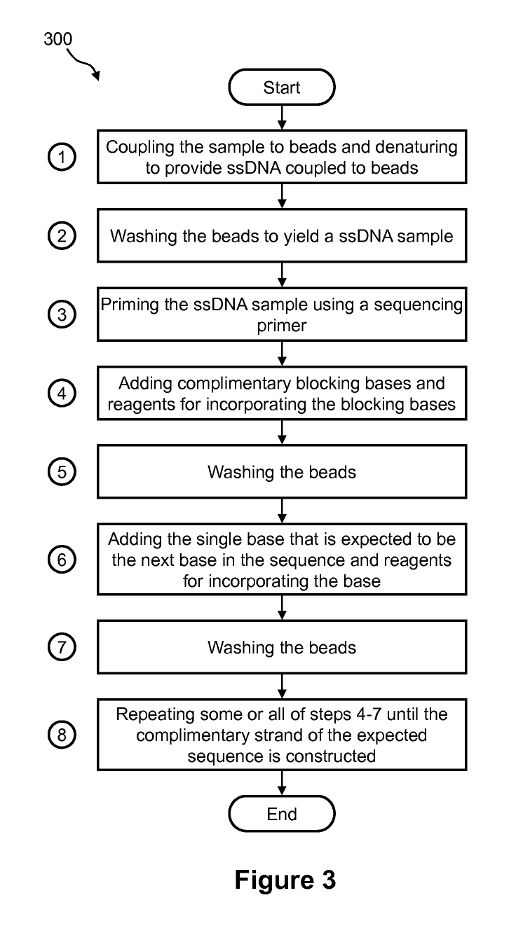

In one embodiment, amplification of DNA occurs using polymerase chain reaction (“PCR”) cycling, which typically includes a heat denaturing step (wherein double stranded target DNA molecules are separated into two single stranded target DNA molecules), an annealing step (wherein oligonucleotide primers complementary to the 3′ boundaries of the target DNA molecules are annealed at low temperature), and a primer extension or elongation step (wherein DNA molecules are synthesized that are complementary to the single stranded target DNA molecules via sequential nucleotide incorporation at the ends of the primers at an intermediate temperature). Typically, one set of these three consecutive steps is referred to as a “cycle.” In one embodiment, a DNA error correction method may be used in a protocol for construction of synthetic DNA (e.g., for construction of synthetic genes). FIG. 1 illustrates a flow diagram of one embodiment of a protocol 100 for DNA synthesis. Protocol Method 100 of DNA synthesis may include, but is not limited to, the following steps:
1. Synthesizing DNA molecules comprising the nucleotide sequence of a template DNA molecule to produce a DNA sample;
2. Performing a DNA error correction method, the method comprising pyrocorrection (e.g., the method 200 of FIG. 2 or the method 300 of FIG. 3) to reduce or eliminate imperfect DNA strands in the DNA sample; and
3. Amplifying the DNA in the DNA sample to increase the quantity of perfect DNA strands in the DNA sample.

FIG. 2 illustrates a flow diagram of one embodiment of a method 200 of pyrocorrection. Method 200 of pyrocorrection may include, but is not limited to, the following steps:
1. Blocking the synthesis of a DNA molecule when the next base to be added during primer extension differs from an expected base as compared to the nucleotide sequence of the template DNA molecule;
2. Adding the expected base to the DNA molecule as compared to the nucleotide sequence of the template DNA molecule; and
3. Repeating steps 1 and 2 until the synthesis of a DNA molecule comprising the nucleotide sequence of a template DNA molecule is complete, and wherein synthesis of a DNA molecule that does not comprise the expected nucleotide sequence as compared to the nucleotide sequence of a template DNA molecule is blocked.

FIG. 3 illustrates a flow diagram of an example of a method 300 of performing DNA error correction according to the present invention (i.e., pyrocorrection). More specifically, method 300 provides more details of an example of the method 200 of pyrocorrection. Method 300 may include, but is not limited to, the following steps:
1. Coupling DNA molecules in a DNA sample to beads;
2. Denaturing the DNA molecules;
3. Washing the beads to yield single stranded DNA molecules coupled to the beads;
4. Annealing primers to the single stranded DNA molecules coupled to the beads;
5. Blocking the synthesis of a DNA molecule when the next base to be added during primer extension differs from an expected base as compared to the nucleotide sequence of a template DNA molecule, wherein blocking the synthesis of a DNA molecule comprises adding complementary blocking bases (e.g., dideoxynucleotides) and reagents for adding the blocking bases during primer extension of the DNA molecule being synthesized, wherein the blocking bases comprise each of the three bases that are not the expected base as compared to the nucleotide sequence of a template DNA molecule;
6. Washing the beads;
7. Adding bases (i.e., deoxynucleotides) and reagents for adding the bases during primer extension of the DNA molecule being synthesized, wherein the bases comprise the expected base as compared to the nucleotide sequence of the template DNA molecule;
8. Washing the beads; and
9. Repeating some or all of steps 5-8 until the synthesis of a DNA molecule comprising the nucleotide sequence of the template DNA molecule is complete.
   At the beginning of methods 200 or 300, the DNA sample will include a mixture of DNA molecules: some perfect (i.e., DNA molecules comprising a nucleotide sequence having 100% sequence identity to the nucleotide sequence of a template DNA molecule), some with errors (i.e., DNA molecules comprising a nucleotide sequence having less than 100% sequence identity to the nucleotide sequence of a template DNA molecule). As the process continues, the synthesis of DNA molecules with errors will be terminated, and only DNA molecules comprising the nucleotide sequence of the template DNA molecule will be fully extended. The DNA molecules comprising the nucleotide sequence of the template DNA molecule may be synthesized with flanking primer sequences for use in amplification methods.

Steps 5 and/or 7 of method 300 (or steps 1 and/or 2 of method 200) may be accomplished using pyrosequencing chemistry. If desired, successful incorporation of the correct or expected base in DNA molecules as compared to the nucleotide sequence of the template DNA molecule may be measured by detecting released PPi. Examples of suitable pyrosequencing chemistry techniques are found in Pollack et al., U.S. Pat. No. 7,727,723, entitled “Droplet-based pyrosequencing,” and Gunderson et al., U.S. Pat. No. 8,486,625, entitled “Detection of nucleic acid reactions on bead arrays,” the entire disclosures of which are incorporated herein by reference.

In one embodiment, within step 5 of method 300 (or step 1 of method 200), the complementary blocking bases comprise dideoxynucleotides. Dideoxynucleotides (also known as 2′,3′ dideoxynucleotides) are chain-terminating inhibitors of DNA polymerase, and are abbreviated as ddNTPs (i.e., ddGTP, ddATP, ddTTP and ddCTP). The absence of a 3′-hydroxyl group means that, after being added by a DNA polymerase to a growing nucleotide chain, no further nucleotides can be added since no phosphodiester bond can be created. Normally, deoxyribonucleoside triphosphate bases (i.e., dGTP, dATP, dTTP and dCTP) allow DNA molecule synthesis to occur through a condensation reaction between the 5′ phosphate of a nucleotide to be added to the DNA molecule being synthesized with the 3′ hydroxyl group of the previous nucleotide. Since dideoxyribonucleotides do not have a 3′ hydroxyl group, no further chain elongation (i.e., primer extension) can occur once a dideoxynucleotide is added to the DNA molecule, which results in termination of synthesis of the DNA sequence.

The washing steps of method 300 may be accomplished as described in Pamula et al., U.S. Pat. No. 7,439,014, “Droplet-based surface modification and washing,” the entire disclosure of which is incorporated herein by reference. The beads may be replaced with any suitable substrate, e.g., a droplet actuator surface, as described in Pamula et al., U.S. Pat. No. 7,439,014, “Droplet-based surface modification and washing.”

The method 200 and/or the method 300 of DNA error correction increases the number of perfect DNA strands (i.e., DNA molecules comprising a nucleotide sequence having 100% sequence identity to the nucleotide sequence of a template DNA molecule) in the DNA sample. For example, the method 300 of DNA error correction can increase the number of perfect DNA strands in the DNA sample by at least 1.5×, 2×, 3×, 4×, or 5×.

In another embodiment, the method 200 and/or the method 300 of DNA error correction (i.e., pyrocorrection) is supplemented by one or more other error correction methods. In one example, an enzyme-surveillance error correction method (i.e., a mismatch-specific DNA endonuclease error correction method) is used. For example, SURVEYOR® Mutation Detection Kits are available from Transgenomic, Inc., Omaha, Nebr. In one example, a gene synthesis protocol may include an enzyme-surveillance error correction method (i.e., a mismatch-specific DNA endonuclease error correction method) and the method 200 and/or the method 300 of pyrocorrection.

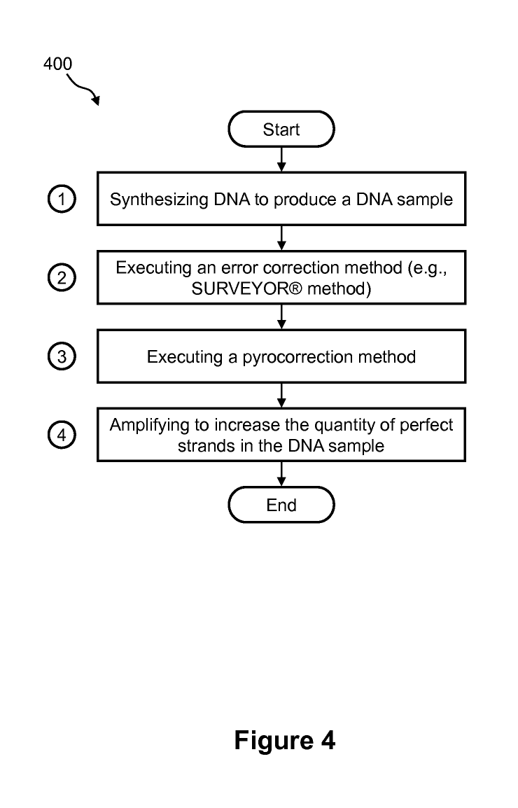

FIG. 4 illustrates a flow diagram of an example of a protocol 400 for gene synthesis that includes an enzyme-surveillance error correction method and the method 300 of pyrocorrection. Protocol 400 may include, but is not limited to, the following steps.
1. Synthesizing DNA molecules comprising the nucleotide sequence of a template DNA molecule to produce a DNA sample;
2. Performing an enzyme-surveillance error correction method, such as a SURVEYOR® method;
3. Performing a DNA error correction method, the method comprising pyrocorrection (e.g., the method 200 of FIG. 2 or the method 300 of FIG. 3) to reduce or eliminate imperfect DNA strands in the DNA sample; and; and
4. Amplifying the DNA in the DNA sample to increase the quantity of perfect DNA strands in the DNA sample.

In another embodiment, the method 200 and/or the method 300 of DNA error correction is supplemented by DNA synthesis methods that employ high fidelity DNA synthesis conditions, such as high fidelity polymerases. Examples include PHUSION® high-fidelity DNA polymerases and Q5® high-fidelity DNA polymerases (both Available from New England Biolabs).

In some embodiments, by using a high fidelity polymerase, the method 200 and/or the method 300 of DNA error correction (i.e., pyrocorrection), and SURVEYOR® methods, the number of perfect DNA strands (i.e., DNA molecules comprising a nucleotide sequence having 100% sequence identity to the nucleotide sequence of a template DNA molecule) can be increased to at least about 90%, at least about 91%, at least about 92%, at least about 93%, at least about 94%, at least about 95%, at least about 96%, at least about 97%, at least about 98%, or at least about 99% of the DNA molecules in the DNA sample.

In some embodiments, for template DNA sequences having from about 100 to about 1000 base pairs, by using a high fidelity polymerase, the method 200 and/or the method 300 of DNA error correction (i.e., pyrocorrection), and SURVEYOR® methods, the number of perfect DNA strands (i.e., DNA molecules comprising a nucleotide sequence having 100% sequence identity to the nucleotide sequence of a template DNA molecule) can be increased to at least about 90%, at least about 91%, at least about 92%, at least about 93%, at least about 94%, at least about 95%, at least about 96%, at least about 97%, at least about 98%, or at least about 99% of the DNA molecules in the DNA sample.

In some embodiments, for template DNA sequences having from about 1000 to about 10,000 base pairs, by using a high fidelity polymerase the method 200 and/or the method 300 of DNA error correction (i.e., pyrocorrection), and SURVEYOR® methods, the number of perfect DNA strands (i.e., DNA molecules comprising the nucleotide sequence of the template DNA molecule) can be increased to at least about 90%, at least about 91%, at least about 92%, at least about 93%, at least about 94%, at least about 95%, at least about 96%, at least about 97%, at least about 98%, or at least about 99% of the DNA molecules in the DNA sample.

In yet another embodiment, the method 200 and/or the method 300 of DNA error correction (i.e., pyrocorrection) makes use of blocking bases (e.g., reversible blocking or terminator bases such as dideoxynucleotides) to account for homopolymeric runs. For example, blocking/deblocking bases may be used for incorporating nucleotides in regions of homopolymeric runs.

In yet another embodiment, instead of single bases, the method 200 and/or the method 300 of DNA error correction (i.e., pyrocorrection) makes use of dinucleotides, trinucleotides, and/or other polynucleotides, rather than mononucleotides.

### 7.2 Determining DNA Size Distribution

In one aspect, the invention provides a method of determining the average size of DNA fragments using pyrophosphate (PPi) release. DNA fragments are incubated with terminal deoxytransferase enzyme and dideoxy ATP. This reaction causes the incorporation of adenosine at both ends of the DNA fragment. During the incorporation reaction, an inorganic phosphate (PPi) is released. This reaction is combined with a pyrophosphatase/luciferase/luciferin mixture to generate a chemiluminescent signal proportional to the amount of PPi released. The PPi-based signal is proportional to the number of nucleic acid ends, and therefore inversely proportional to the sample's average fragment size. The concentration of the DNA sample (in ng/uL) is converted to molarity to reflect the number of molecules present in the sample. The molarity is then divided by half the total PPi signal detected (which should relate to the number of molecules), and again divided by 660 (the molecular weight of a single nucleotide base pair) to arrive at average fragment size.

In one example, the invention provides a method of calculating DNA size using terminal deoxytransferase, pyrophosphate determination, and DNA concentration.

In one embodiment, the invention provides a method of determining the average size of DNA fragments in a DNA sample, the method comprising:
1. conducting a pyrosequencing reaction comprising combining the DNA sample and pyrosequencing reagents, wherein the pyrosequencing reaction is conducted without determining the nucleic acid sequences of the DNA fragments in the DNA sample, whereby the pyrosequencing reaction yields a detectable pyrophosphate concentration;
2. determining the pyrophosphate concentration; and
3. determining the average size of DNA fragments in the DNA sample based on the pyrophosphate concentration.

Combining the DNA sample and pyrosequencing reagents may include incubating the DNA sample with terminal deoxytransferase and ddATP, wherein dideoxynucleotides are incorporated into the DNA fragments in the DNA sample. The pyrophosphate concentration in the DNA sample may be determined in moles/liter, and particularly may be determined by performing a chemiluminescence assay on the DNA sample. Determining a DNA concentration in the DNA sample may include performing qPCR on the DNA sample. In addition, determining the average size of DNA fragments in the DNA sample based on the pyrophosphate concentration may comprise the steps of:
i. determining a DNA concentration in the DNA sample in grams/liter;
ii. calculating the average molecular weight of the DNA fragments in the DNA sample in grams/mole, comprising dividing the DNA concentration in grams/liter by ½ the pyrophosphate concentration in moles/liter; and
iii. calculating the average size of DNA fragments in the DNA sample, comprising dividing the average molecular weight of DNA in grams/mole by 660 grams/base pair.

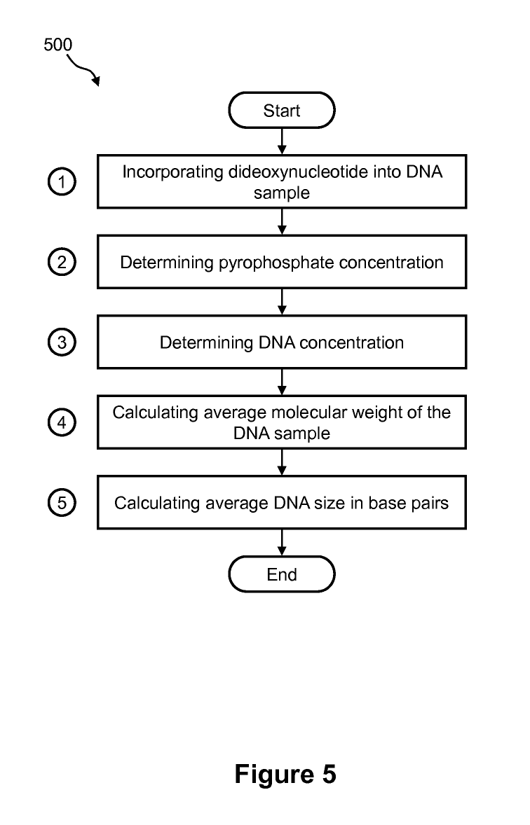

FIG. 5 illustrates a flow diagram of an example of a method 500 of calculating the average size of DNA fragments. Method 500 may include, but is not limited to, the following steps:
1. Incorporating dideoxynucleotides into the DNA molecules in a DNA sample (e.g., incorporating dideoxynucleotides into the DNA molecules in the DNA sample using terminal deoxytransferase and ddATP, which release 2 pyrophosphate molecules for every DNA molecule);
2. Determining the pyrophosphate concentration in the DNA sample (e.g., determining the pyrophosphate concentration in moles/liter using a pyrophosphate chemiluminescence assay;
3. Determining the DNA concentration in the DNA sample (e.g., determining the concentration of DNA in grams/liter in the sample by qPCR, such as PicoGreen®, EvaGreen®, NuPCR techniques, and the like);
4. Calculating the average molecular weight of DNA in the DNA sample (e.g., calculating the average molecular weight of DNA in grams/mole by dividing the DNA concentration in grams/liter by ½ the pyrophosphate concentration in moles/liter); and
5. Calculating the average size of DNA fragments in the DNA sample (e.g., calculating the average number of base pairs per DNA molecule by dividing the average molecular weight of DNA in grams/mole by 660 grams/base pair).

### 7.2.1 Testing for Nucleotide Repeat Disorders

The method of determining the average size of DNA fragments is useful as a method for diagnosing or screening for nucleotide repeat disorders, such as trinucleotide repeat disorders. Examples include polyglutamine diseases, such as dentatorubropallidoluysian atrophy, Huntington's disease, spinobulbar muscular atrophy, spinocerebellar ataxia types 1, 2, 3, 6 and 7, as well as non-polyglutamine diseases, such as fragile X Syndrome, fragile XE mental retardation, Friedreich's Ataxia, myotonic dystrophy, and spinocerebellar ataxia types 8 and 12.

In one embodiment, the invention provides a method of diagnosing, screening, confirming, or identifying individuals with fragile X syndrome, or permutation carriers of fragile X syndrome. Fragile X syndrome is caused by an expansion mutation in the Fragile X mental retardation 1 (FMR1) gene (NCBI Gene ID: 2332). In fragile X, the FMR1 gene includes a repetitive CGG trinucleotide sequence in its 5′ untranslated region (UTR). CGG is repeated six to 50 times in unaffected persons. A full FMR1 mutation includes more than 200 CGG repeats in the FMR1 gene and hypermethylation, which leads to an inability to produce the FMR1 protein. Permutation carriers have between about 55 and about 200 CGG repeats, called permutations. Permutation carriers are susceptible to developing premature ovarian failure and Fragile X-associated tremor/ataxia syndrome (FXTAS). In some cases, the fragile X phenotype occurs in a permutation carrier if hypermethylation is present. About 40 to about 55 repeats is considered a “grey zone” where normal and permutation size ranges overlap. Expansions with more than 200 repeats, called full mutations, are associated with increased methylation of that region of the DNA which effectively silences the expression of the FMR1 protein. During screening for Fragile X syndrome, the number of CGG repeats is measured to assess the severity of the disease. One of the challenges in screening procedures for Fragile X syndrome is to determine the number of CGG repeats in a heterologous individual that vary in number.

In one embodiment, the FMR1 gene or relevant portion of the FMR1 gene (e.g., the 5′ untranslated region) is amplified. qPCR is used to measure the total amount of nucleotides in the sample. The determining DNA size distribution technique of the invention is used to measure the number of nucleic acid molecules in the sample. The ratio of the total nucleotides to the quantity of nucleotides is used to determine the number or approximate number of CGG repeats. The number of CGG repeats in the amplified FMR1 gene or relevant portion of the amplified FMR1 gene is then used to determine whether the individual: (1) has fragile X; (2) is a permutation carrier of fragile X; (3) is within the normal range; or (4) some other condition. Of course, the ranges given above are subject to reinterpretation or reclassification by the medical community as more is learned about the implications of the mutation. The invention provides an easy means for quickly identifying the lengths of the mutations, which may then be interpreted diagnostically. Diagnosis, screening, confirming, or identifying may also include interpreting the results of the determining DNA size distribution test together with other diagnostic tests and information (such as phenotypical traits). A similar approach may be used for other nucleotide repeat disorders.

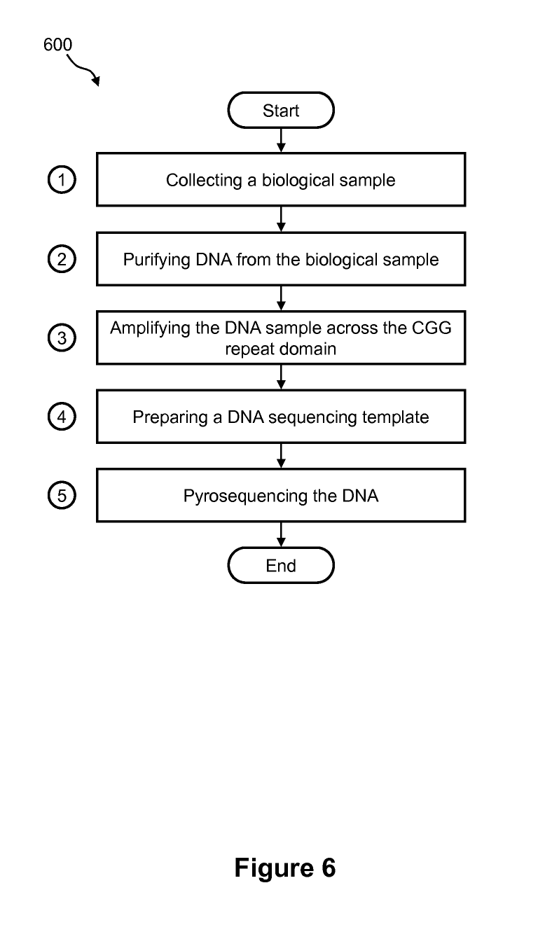

FIG. 6 illustrates a flow diagram of an example of a method 600 for diagnosing or screening for nucleotide repeat disorders. Method 600 may include, but is not limited to, the following steps:
1. Collecting a biological sample (e.g., a cheek swab or a dried blood spot);
2. Purifying DNA from the biological sample;
3. Amplifying the DNA across the CGG repeat domain of the FMR1 gene (e.g., the 5′ untranslated region of the FMR1 gene);
4. Preparing a DNA template; and
5. Pyrosequencing the DNA.

Accordingly, in one embodiment, the invention provides methods for droplet-based genotyping assays for enumeration of CGG trinucleotide repeats in the FRM1 gene. The genotyping assays combine protocols for sample preparation, PCR amplification, template preparation, and pyrosequencing of the CGG trinucleotide repeat domain on a single droplet actuator. In one embodiment, the genotyping assay may use the nucleotide natural block method for pyrosequencing. The amount of light normally generated by pyrosequencing is proportional to the number of adjacent unpaired bases complementary to the added nucleotide. However, in repeated or homopolymeric regions of DNA it is often difficult to decipher the sequence of the growing DNA strand. In the nucleotide natural block method, blocking nucleotides are used to temporarily terminate the polymerase reaction. Because the polymerase reaction is blocked by incorporation of the nucleotide analog, only one nucleotide is incorporated during a reaction cycle. Sequencing is performed by alternating the presentation of dCTP or dGTP nucleotides until the blocking nucleotides are reached. The nucleotide natural block method identifies the 3′-end of the CGG repeats and provides a count of the number of CGG repeats in both alleles.

In another embodiment, the genotyping assay includes a PCR amplification protocol that incorporates uracil during amplification of the CGG repeat domain.

On-bench protocols for each step of the genotyping assays may be adapted and described as discrete step-by-step, droplet-based protocols. Protocol steps are performed in aqueous droplets within an oil-filled gap of a droplet actuator. Samples and assay reagents are manipulated as discrete droplets upon an arrangement of electrodes (i.e., digital electrowetting). Sample droplets and reagent droplets for use in conducting the various protocol steps may be dispensed and/or combined according to appropriate assay protocols using droplet operations on a droplet actuator. Incubation and washing of assay droplets, including temperature adjustments as needed, may also be performed on a droplet actuator. Further, detection of signals from assay droplets, such as detection of fluorescence may be conducted while the droplet is present on the droplet actuator. Further, each of these processes may be conducted while the droplet is partially or completely surrounded by a filler fluid on the droplet actuator.

In one step, a biological sample is collected and transferred to a sample preparation reservoir of a droplet actuator. In one embodiment, the biological sample is a cheek cell sample obtained via a buccal swab. In another embodiment, the biological sample is a dried blood spot sample. DBS samples may, for example, be prepared from blood samples collected and dried on filter paper. A manual or automatic puncher may be used to punch a sample, e.g., a 3 mm punch. The sample preparation reservoir may contain a fluid that is used to resuspend the sample and release the cells into the solution.

In another step, genomic DNA in the biological sample is isolated, purified and concentrated in a sample preparation module integrated on the droplet actuator. In one example, genomic DNA, such as genomic DNA from blood cells, may be prepared using magnetically responsive beads (e.g., Dynabeads DNA DIRECT from Dynal). A droplet including lysis buffer and magnetically responsive beads may be combined using droplet operations with a blood sample to yield a DNA capture droplet in which released DNA is bound to the beads. The DNA capture droplet may be transported using droplet operations into the presence of a magnet and washed using a merge-and-split wash protocol to remove unbound material, yielding a washed DNA capture droplet substantially lacking in unbound material. A droplet including resuspension buffer may be merged with the washed DNA capture droplet. The DNA capture droplet may be transported using droplet operations into a thermal zone to promote release of DNA from the beads, e.g., by heating to approximately 65° C. The eluted DNA contained in the droplet surrounding the beads may then be transported away from the beads for further processing on the droplet actuator, e.g., for execution of a droplet based PCR amplification protocol.

In another step, target nucleic acid sequences (i.e., CGG trinucleotide repeat domain) are amplified in a PCR module integrated on the droplet actuator. In this step, primers flanking the FMR1 CGG trinucleotide repeat domain are used for amplification. To provide a platform for subsequent digital microfluidic pyrosequencing, one of the PCR primers may be a 5′-biotinylated primer. The 5′-biotinylated primer provides a ready method for anchoring the sequencing template DNA strand to magnetically responsive beads, such as streptavidin-coated magnetic beads. A droplet including PCR reagents (e.g., dNTPs, enzyme, primers) may be combined using droplet operations with a DNA sample droplet to yield a reaction droplet. PCR amplification may, for example, be performed in a flow-through format where for each cycle the reaction droplets are cyclically transported using droplet operations between different temperature zones (e.g., 95° C. zone and a 55° C. zone) within the oil filled droplet actuator. To remove excess biotinylated primers from the reaction droplet, a droplet including wash buffer and magnetically responsive beads (e.g., Dynabeads DNA DIRECT from Dynal) may be combined using droplet operations with a reaction droplet to yield a DNA capture droplet. The DNA capture droplet may be transported using droplet operations into the presence of a magnet and washed using a merge-and-split wash protocol to remove unbound material. The washed DNA capture droplet may be transported using droplet operations into a thermal zone to promote release of DNA from the beads, e.g., by heating to approximately 65° C. The eluted DNA contained in the droplet surrounding the beads may then be transported away from the beads to yield an eluted DNA droplet. A droplet including streptavidin-coated magnetically responsive beads may be merged with the eluted DNA droplet, yielding an amplified DNA/bead-containing droplet. The amplified DNA/bead-containing droplet may be transported using droplet operations into a thermal zone (e.g., about 65° C.) for a period of time sufficient to promote formation of biotin-streptavidin complexes. The biotinylated PCR amplicons are immobilized on the beads through formation of biotin-streptavidin complexes.

In another step, amplified sequences are prepared for pyrosequencing in a template preparation module integrated on the droplet actuator. In one example, single stranded sequencing template is prepared by alkali denaturation. An example of a process of preparing a single stranded template for pyrosequencing on a droplet actuator is as follows. An amplified DNA/bead-containing droplet is washed using a merge-and-split protocol with a reagent droplet that contains a denaturation solution (e.g., 0.5 M sodium hydroxide (NaOH)). After washing, the amplified DNA/bead-containing droplet is merged with a second reagent droplet and incubated at ambient temperature for a period of time sufficient to denature DNA. The amplified DNA/bead-containing droplet that now has single-stranded DNA (ssDNA) bound therein is transported using droplet operations into the magnetic field of a magnet. A first bead washing protocol is used to exchange the denaturation solution in the ssDNA/bead-containing droplet with a wash buffer. A second washing protocol is used to exchange the wash buffer in the ssDNA/bead-containing droplet with an annealing buffer. The ssDNA/bead-containing droplet is combined using droplet operations with a primer droplet to yield a ssDNA template droplet. The ssDNA template droplet is incubated at an annealing temperature (e.g., about 80° C.) for a period of time (e.g., about 2 minute) sufficient for annealing of primer to ssDNA template. After the incubation period, a bead washing protocol is used to remove excess unbound primers from the ssDNA template droplet. In one example, the ssDNA template droplet is washed twice using pyrosequencing buffer droplets. The ssDNA template droplet in pyrosequencing buffer is ready for sequencing.

In another step, the prepared ssDNA template immobilized on magnetically responsive beads is sequenced in a pyrosequencing module integrated on the droplet actuator. An example of a three-enzyme pyrosequencing protocol is as follows. A ssDNA template droplet may be combined with a droplet of one of the four nucleotides mixed with APS and luciferin in wash buffer. A droplet containing all three enzymes (DNA polymerase, ATP sulfurylase and luciferase) may be combined with the merged ssDNA template droplet and nucleotide-containing droplet to yield a reaction droplet. The reaction droplet may be mixed and transported to a detector location. Incorporation of the nucleotide may be detected as a luminescent signal proportional to the number of adjacent bases incorporated into the strand being synthesized, or as a background signal for a non-incorporated (mismatch) nucleotide. After the reaction is complete, the reaction droplet may be transported to a magnet and washed. Washing may be accomplished by addition and removal of wash buffer while retaining substantially all beads (with bound template thereon) in the droplet. This entire sequence constitutes one full pyrosequencing cycle which may be repeated multiple times with a user defined sequence of base additions.

### 7.3 Determining Library Size Distribution and Bias

The invention provides a method of using pyrosequencing to determine size distribution and bias in a library of nucleic acids. In one embodiment, the method of the invention may be used to readily determine the quality of a library that may be used in a next-generation sequencing protocol.

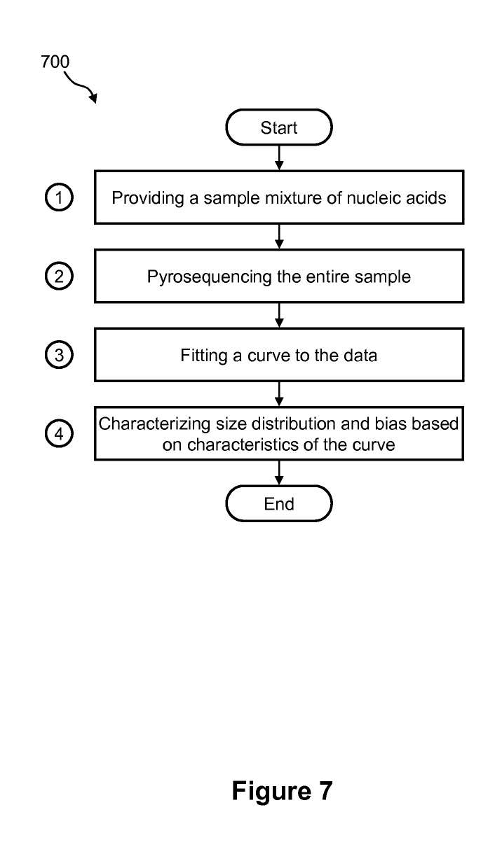

FIG. 7 illustrates a flow diagram of a method 700 of determining size distribution and bias in a DNA library. Method 700 may include, but is not limited to, the following steps:
1. Providing a DNA sample;
2. Pyrosequencing DNA molecules in the DNA samples, thereby producing pyrosequencing data;
3. Fitting a curve to the pyrosequencing data; and
4. Characterizing library size distribution and bias based on characteristics of the curve.

In one example, step 2 of method 700 may be accomplished by sequentially incorporating first a mixture of dATP and dTTP, followed by a mixture of dGTP and dCTP. The process may be repeated until complementary strands in the DNA sample are completely synthesized. Pyrosequencing chemistry is known in the art. In this and other embodiments of the invention, known pyrosequencing chemistry can be used, including without limitation, the chemistry described in Pollack et al., U.S. Pat. No. 7,727,723, entitled “Droplet-based pyrosequencing,” and Gunderson et al., U.S. Pat. No. 8,486,625, entitled “Detection of nucleic acid reactions on bead arrays,” the entire disclosures of which are incorporated herein by reference.

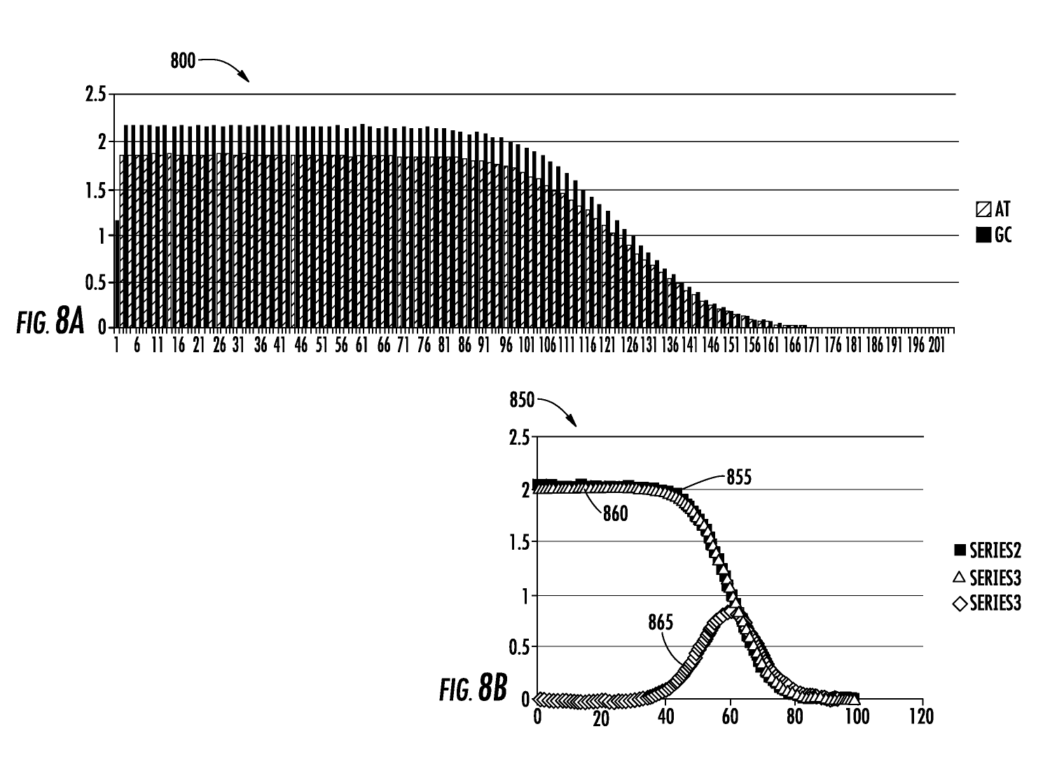

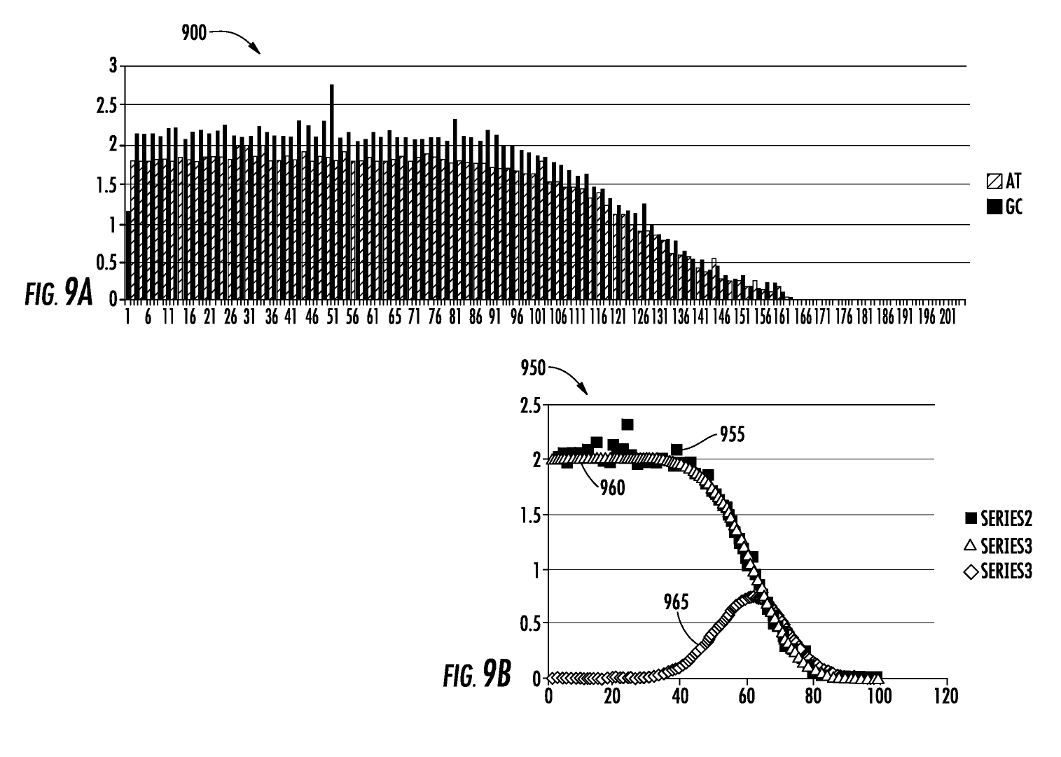

A five-parameter nonlinear fit of the pyrosequencing data is used to generate output data that is used to characterize the library. Output data includes GC-content (i.e., % GC), standard deviation of AT-content, standard deviation of GC-content, average fragment size, and standard deviation of fragment size. GC-content is used as an indicator that the library comprises the appropriate DNA. Standard deviations of AT-content and GC-content are used as an indicator of bias in the library. Library bias is calculated to show that no single sequence or set of sequences are over-represented in the library. In a library with no bias or very little bias, the standard deviation of GC-content and AT-content is low. In a library with bias, the standard deviation of GC-content and AT-content is higher relative to a library with no bias. The average size of fragments in the library is calculated to determine if the fragments in the library are of suitable size. The standard deviation of fragment size is calculated to determine if the distribution of fragments in the library is within a suitable range. In one example, a derivative of the curve is used as a representation of the size distribution of the library. Examples of pyrosequencing data analysis for characterizing a nucleic acid library are described with reference to FIGS. 8A and 8B and FIGS. 9A and 9B.

FIGS. 8A and 8B show a pyrogram 800 and a plot 850, respectively, of pyrosequencing results and analysis of a simulated non-biased nucleic acid library. Pyrogram 800 of FIG. 8A is the pyrogram output of the simulated non-biased library. The input parameters for the simulated non-biased library are shown in Table 1.

TABLE 1
Simulated non-biased library input

| Input | Parameter | Range |
|---|---|---|
| 46.1% | GC % | (0.1-99.8) |
| 250 | Fragment Length | (1-5000) |
| 35 | StDev Frag Length | (1-2000) |
| 0% | Bias Fragment 1% | (0-99) |
| 0% | Bias Fragment 2% | (0-99) |
| 30000 | Fragments | |
| 10000 | Read Limit | |

Plot 850 of FIG. 8B is a plot of the analysis of the pyrogram 800 of FIG. 8A. Plot 850 is used to generate the output data of the simulated non-bias library. Plot 850 shows a curve 855 (Series 2) that plots the average of A, T, G, C chemiluminescent signals for each incorporation in the pyrosequencing reaction. Plot 850 also shows a curve 860 (Series 3) that plots a five-parameter nonlinear fit of the data in curve 855 (Series 2). Plot 850 also shows a curve 865 that plots the derivative of curve 860. Curve 860 is used to determine the observed GC %. The standard deviations (StDev) of GC-content and AT-content in the flat region of curve 860 are used to determine the bias in the library. For example, an algorithm is used to convert the standard deviations of GC-content and AT-content to a prediction of percent bias using an empirical data set. In a library with no bias or very little bias the standard deviations of GC and AT in the flat region of curve 860 are low. Full-width at half-maximum (FWHM) of curve 865 is, for example, used to determine the fragment size distribution of the library. Table 2 shows the calculated output data obtained from plot 850.

TABLE 2
Output data of simulated non-biased library

| Output | Parameter |
|---|---|
| 46.1% | GC % |
| 0.096 | StDev AT in flat region |
| 0.082 | StDev GC in flat region |
| 246 | Calculated average fragment length |
| 40 | Calculated StDev of fragment length |

FIGS. 9A and 9B show a pyrogram 900 and a plot 950, respectively, of pyrosequencing results and analysis of a simulated biased nucleic acid library. Pyrogram 900 of FIG. 9A is the pyrogram output of the simulated biased library. The input parameters for the simulated biased library are shown in Table 3.

TABLE 3
Simulated biased library input

| Input | Parameter | Range |
|---|---|---|
| 46.1% | GC % | (0.1-99.8) |
| 250 | Fragment Length | (1-5000) |
| 35 | StDev Frag Length | (1-2000) |
| 5% | Bias Fragment 1% | (0-99) |
| 0% | Bias Fragment 2% | (0-99) |
| 20000 | Fragments | |
| 10000 | Read Limit | |

Plot 950 of FIG. 9B is a plot of the analysis of the pyrogram 900 of FIG. 9A. Plot 950 shows a curve 955 (Series 2) that plots the average of A, T, G, C chemiluminescent signals for each incorporation in the pyrosequencing reaction. Plot 950 also shows a curve 960 (Series 3) that plots a five-parameter nonlinear fit of the data in curve 955 (Series 2). Plot 950 also shows a curve 965 that plots the derivative of curve 955. The observed GC-content (i.e., GC %) are determined from curve 960. The standard deviations (StDev) of GC-content and AT-content in the flat region of curve 960 are used to determine the bias in the library. In a library with bias, the standard deviations of GC and AT in the flat region of curve 960 are higher relative to a library with no bias. Full-width at half-maximum (FWHM) of curve 965 is, for example, used to determine the fragment size distribution of the library. Table 4 shows the calculated output data obtained from plot 950.

TABLE 4
Output data of simulated biased library

| Output | Parameter |
|---|---|
| 46.1% | Observed GC % |
| 0.114 | StDev AT in flat region |
| 0.104 | StDev GC in flat region |
| 254 | Calculated average fragment length |
| 48 | Calculated StDev of fragment length |

### 7.4 Pyrosequencing Read Length

The preferred substrate for firefly luciferase is ATP; however, dATP is also readily utilized by luciferase which in the presence of luciferin results in the production of light. The use of dATP for nucleotide incorporation in pyrosequencing gives rise to a substantial signal which would be interpreted either as a large background signal or a false positive A incorporation. To improve the pyrosequencing reaction and eliminate the problems associated with the use of dATP, dATP may be replaced by 2′-deoxyadenosine-5′-O′-1-thiotriphosphate (dATP-α-S); dATP-α-S is incorporated by DNA polymerases but is poorly utilized by luciferase. The substitution of dATP-α-S for dATP will produce a lower background signal; however, the use of dATP-α-S in pyrosequencing is not without negative consequences. For example, the incorporation of dATP-α-S by Klenow DNA polymerase may be less than 100% efficient leading to unequal nucleotide incorporation especially in A-rich sequences or sequences containing homopolymeric runs of A's. With increasing incorporation of dATP-α-S in the growing strand, the sequence downstream will become more and more asynchronous resulting in uneven signals and a uniform decrease in signal peak heights, partially slipping out of phase and leading to ambiguous sequence data with increasing read lengths.

Several approaches may be used to improve pyrosequencing quality and read length in pyrosequencing reactions. In one embodiment, the various approaches to improve pyrosequencing quality and read length may be used in pyrosequencing reactions performed on a droplet actuator. In one example, luciferase may be replaced with a modified luciferase that poorly utilizes or cannot utilize dATP. Several modified luciferases have been created by site directed mutagenesis which reduced the ability of luciferase to utilize dATP from about 4 fold to about 160 fold. The use of a modified luciferase allows the replacement of dATP-α-S by the natural dATP during the nucleotide incorporation phase. The use of a modified luciferase and dATP may be used to improve the sequence output without increasing the background signal.

In another example, dATP-α-S may be replaced with another modified adenosine nucleotide. The modified adenosine nucleotide may be selected such that the modified nucleotide is a suitable substrate for the DNA polymerase, but is a substantially poor substrate or a non-substrate for luciferase.

In yet another example, dATP-α-S may be replaced with dATP during the nucleotide incorporation phase of the pyrosequencing assay, but degrade all unincorporated dATP before initiating the detection phase of the assay. In this approach, a step that uses an enzyme or enzymes that degrade dATP, but leaves pyrophosphate untouched, may be incorporated into the pyrosequencing assay before initiating the detection phase of the assay. In one example, apyrase may be used to degrade dATP and leave pyrophosphate untouched since pyrophosphate is not a substrate of apyrase.

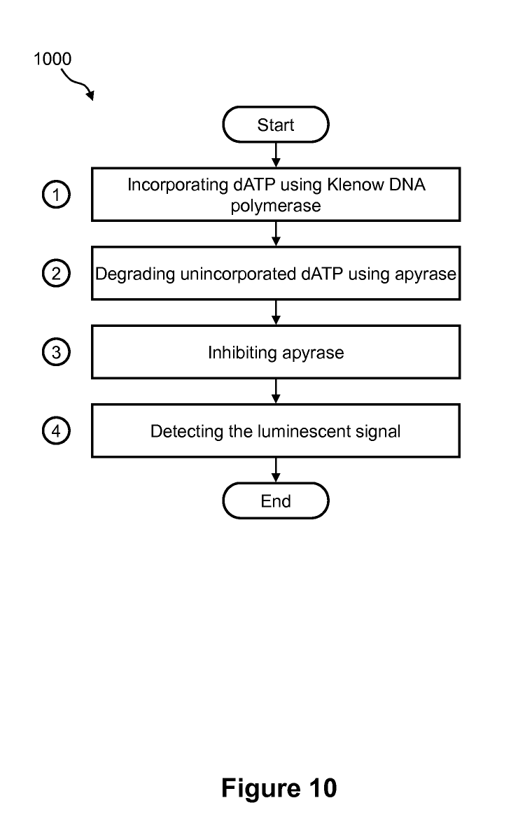

FIG. 10 illustrates an example of a method 1000 of using dATP and apyrase in a pyrosequencing assay performed on a droplet actuator. Method 1000 may include, but is not limited to, the following steps:
1. Incorporating dATP using Klenow DNA polymerase (e.g., a DNA template droplet is combined using droplet operations with a reaction droplet that includes dATP and DNA polymerase to yield a reaction droplet, wherein the reaction generates pyrophosphate and unused excess dATP);
2. Degrading unincorporated dATP using apyrase (e.g., the reaction droplet is split using droplet operations to yield two reaction droplets, wherein one reaction droplet is combined using droplet operations with a droplet containing apyrase in solution to yield a reaction/apyrase droplet, further wherein the apyrase degrades all unincorporated dATP);
3. Inhibiting apyrase (e.g., a droplet containing sodium azide or sodium fluoride is combined using droplet operations with the reaction/apyrase droplet to yield a dATP-free droplet, wherein the sodium azide or sodium fluoride inhibits apyrase);
4. Detecting a luminescent signal (e.g., the dATP-free droplet is combined using droplet operations with a droplet that contains sulfurylase and luciferase, wherein pyrophosphate in the dATP-free droplet is converted to ATP and then to light, whereby a detectable luminescent signal is produced).

### 7.5 Gene Synthesis

A gene synthesis protocol may be performed on a droplet actuator. In one example, an enzyme-mediated synthesis method may be used to construct a synthetic gene sequence. Briefly, a set of synthetic oligonucleotides (e.g., 6 oligonucleotides, 60 nucleotides in length) are designed for a region of a DNA molecule of interest such that the ends of each oligonucleotide overlap other oligonucleotides in the set. The oligonucleotides are assembled into individual “cassettes” that are a few hundred base pairs in length (e.g., 360 bp).

The oligonucleotides used to construct synthetic genes are typically synthesized by automated machines using phosphoramidite synthesis chemistry. This synthesis process is prone to producing oligonucleotides that contain errors (e.g., deletion errors). As the length of the oligonucleotide sequences are increased, the probability of the oligonucleotides containing errors is also increased. Because the oligonucleotides used to construct a DNA sequence may contain errors, the resulting pool of synthesized DNA strands may also contain errors.

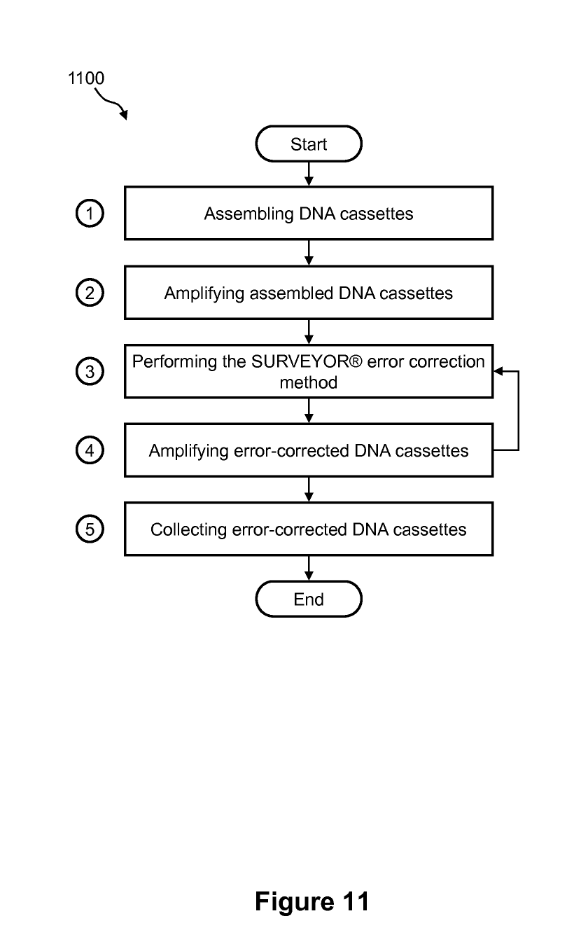

FIG. 11 illustrates a flow diagram of an example of a protocol 1100 for synthesis of a DNA molecule on a droplet actuator. Protocol 1100 uses a set of synthetic oligonucleotide sequences designed for a DNA molecule of interest and PCR cycling to generate a pool of synthesized DNA strands. An error correction method, such as the SURVEYOR® method, is used to increase the quantity of perfect DNA strands in the pool. Protocol 1100 includes, but is not limited to, the following steps:
1. Assembling DNA cassettes. For example, a set of short oligonucleotides (e.g., 6 oligonucleotides of about 60 nucleotides in length) are designed for a region of a DNA molecule of interest such that the ends of each oligonucleotide overlap other oligonucleotides in the set to form a 360 bp fragment. An aliquot (50 μL) of the set of oligonucleotide sequences is transferred to a sample input reservoir of a droplet actuator. The concentration of each oligonucleotide in the set is, for example, from about 200 nM to about 500 nM. A 1× oligonucleotide droplet is dispensed and combined using droplet operations with a 2× assembly reagent droplet to yield a 3× assembly droplet. The 2× assembly reagent droplet includes reagents (e.g., enzymes, dNTPS and buffer) for cassette assembly. The 3× assembly droplet is transported using droplet operations to a temperature control zone on the droplet actuator. The 3× assembly droplet is incubated at 50° C. for about 30 min to about 60 min to assemble the DNA cassettes. The 3× assembly droplet is diluted 1:10 using a droplet dilution protocol to yield a diluted DNA cassette droplet.
2. Amplifying assembled DNA cassettes. For example, a 1× diluted DNA cassette droplet is combined using droplet operations with a 1×PCR reagent droplet that includes polymerase and reagents (e.g., primers, dNTPS, and buffer) for PCR amplification of the assembled DNA cassettes. PCR cycling may, for example, be performed in a flow-through format where for each cycle the DNA assembly droplet is cyclically transported using droplet operations between different temperature zones (e.g., between a 98° C. zone, a 60° C. zone, and a 72° C. zone) within the oil filled droplet actuator. In one example, the PCR cycling is performed using a hot start at 98° C. for 60 sec followed by 24 cycles of 98° C. for 10 sec, 60° C. for 30 sec, and 72° C. for 30 sec; followed by a hold at 72° C. for 5 min. To remove excess PCR reagents (e.g., primers, dNTPS, and salts), the amplified DNA cassettes may be coupled to magnetically responsive beads, such as SPRI beads, and then washed using a bead washing protocol. The washed DNA is eluted from the beads and a 2X washed DNA cassette droplet is transported using droplet operations to a temperature control zone on the droplet actuator to prepare the DNA cassettes for error correction.
3. Performing the SURVEYOR® error correction method. For example, The 2× washed DNA cassette droplet from step 2 is first denatured at 98° C. for 2 min and then annealed by slowly cooling the reaction mixture to 85° C. at a rate of 2° C./min, holding at 85° C. for 2 min, slowly cooling the reaction mixture to 25° C. at a rate of 0.1° C./sec and holding at 25° C. for 2 min. The 2×-DNA-cassette-droplet is then held at 10° C. The 2×-DNA-cassette-droplet is combined using droplet operations with a 1× SURVEYOR® nuclease droplet to yield a 3×-DNA-cassette-droplet. The 3×-DNA-cassette-droplet is combined using droplet operations with a 1× Exonuclease III droplet to yield a 4×-DNA-cassette-droplet. The 4×-DNA-cassette-droplet is incubated at 42° C. for 60 min to cleave the DNA at any gaps created by mismatches in the DNA.
4. Amplifying the error-corrected DNA cassettes. For example, a 1×-DNA-cassette-droplet is combined using droplet operations with a 1×-PCR-reagent-droplet to yield a 2×-amplification-droplet. PCR cycling is performed using a hot start at 98° C. for 60 sec followed by 24 cycles of 98° C. for 10 sec, 60° C. for 30 sec, and 72° C. for 30 sec; followed by a hold at 72° C. for 5 min. Steps 3 and 4 of protocol 1100 are repeated. Then, protocol 1100 proceeds to step 5.
5. Collecting the error-corrected 360 by DNA cassettes.

In subsequent processing steps (not shown), sets of overlapping 360 by DNA cassettes may be assembled into longer molecules. Overlapping bases at each end of the DNA cassettes allow for subsequent assembly of multiple cassettes into longer molecules, through an iterative process, until the desired length (e.g., 2000 by or more) of the DNA molecule is reached.

In another example, a gene synthesis protocol may combine the pyrocorrection method 300 of the invention and the SURVEYOR® error correction method as described with reference to FIG. 4.

### Example

### Determining DNA Size by Pyrophosphate Release (Pyrosizing)

Method 500, described with reference to FIG. 5, of determining the average size of DNA fragments by pyrophosphate release may be performed on a droplet actuator. In this example, the DNA samples were the 1204 bp, and 688 bp fragments from a lambda Hind III digest. DNA samples were amplified and purified on-bench and subsequently loaded into liquid dispensing reservoirs of the droplet actuator. All reagents required for determining DNA size by pyrophosphate release were prepared on-bench and subsequently loaded into liquid dispensing reservoirs of a droplet actuator.

Reagents used to determine DNA size by pyrophosphate release included: Tris acetate, agarose gel, luciferin, magnesium acetate, DL-dithiothreitol (DTT), ethylenediaminetetraacetic acid (EDTA) and Tween 20 were all obtained from Sigma-Aldrich Corp. (St. Louis, Mo.). ddATP was purchased from Fluka (Sigma-Aldrich, St. Louis, Mo.). NTPs were purchased from Kapa (Boston, Mass.). Terminal deoxytransferase, cobalt chloride and 10× buffer were from New England Biolabs (Ipswich, Mass.). ATP sulfurylase (ATPS) was from Biolog (Hayward, Calif.). Luciferase was purchased from Promega (Madison, Wis.). Molecular grade water was obtained from Fisher Scientific (Pittsburgh, Pa.). 5 cSt silicone oil was obtained from Gelest (Morrisville, Pa.). The gel purification column was obtained from Invitrogen (Grand Island, N.Y.). DNA samples were from a lambda Hind III digested PCR amplicon.

The droplet actuator cartridge used in this experiment included one large liquid dispensing reservoir and 16 smaller liquid dispensing reservoirs. In this example, samples and reagents were loaded individually into one the 16 smaller reservoirs.

### 8.1 Detection of Purified PPi on Cartridge (Generation of PPi Standard Curve)

A standard curve was generated using purified PPi to determine the range of PPi detection (in the presence of a ddATP background) of the digital microfluidic platform. A 20 μL enzyme mix was prepared by diluting stock concentrations of ATP sulfurylase (ATPS), luciferase and D-luciferin with buffer and water to final reservoir concentrations as indicated in Table 1. A 20 μL reagent mix was prepared by diluting varying concentrations of PPi (stock concentrations of 20, 10, 5, 2.5, 1.25, 0.625, and 0.3125 μM), APS, DTT and ddATP in buffer and water to final reservoir conditions as indicated in Table 1.

TABLE 1
Reaction mixes for determination of PPi standard curve.

| Reaction | | |
|---|---|---|
| Mix | Stock Concentrations | Reservoir Concentrations |
| Enzyme | ATPS (300 mU/μL) | ATPS (22.5 mU/μL) |
| mix | Luciferase (13.6 μg/μL) | Luciferase (3 μg/μL) |
| | D-luciferin (5 μg/μL) | D-luciferin (2.4 μg/μL) |
| | Tris-acetate, pH 7.6 (1M) | Tris-acetate, pH 7.6 (100 mM) |
| | EDTA, pH 8.0 (10 mM) | EDTA, pH 8.0 (0.5 mM) |
| | Mg acetate (100 mM) | Mg acetate (5 mM) |
| | Tween 20 (1%) | Tween 20 (0.02%) |
| Reagent | PPi (20, 10, 5, 2.5, 1.25, | PPi (2.4, 1.2, 0.61, 0.31, |
| mix | 0.625, 0.3125, and 0 μM) | 0.15, 0.075, 0.0375, and 0 μM) |
| | APS (200 μM) | APS (10 μM) |
| | DTT (25 mM) | DTT (1 mM) |
| | Tris acetate, pH 7.6 (1M) | Tris acetate, pH 7.6 (100 mM) |
| | EDTA, pH 8.0 (10 mM) | EDTA, pH 8.0 (0.5 mM) |
| | Mg acetate (100 mM) | Mg acetate (5 mM) |
| | ddATP (10 mM) | ddATP (0.1 mM) |
| | Tween 20 (0.2%) | Tween 20 (0.02%) |

The reaction mixtures (20 μL each; the enzyme mix and a reagent mix for each concentration of PPi) were loaded into separate liquid dispensing reservoirs of a disposable digital microfluidic cartridge. For each PPi concentration, one droplet (˜100 nL) of the appropriate reagent mix and one droplet of the enzyme mix were dispensed and combined using droplet operations. The combined droplet was mixed briefly at room temperature using droplet operations, and the resulting chemiluminescent signal was detected.

FIG. 13 shows a plot 1300 of a standard curve for the detection of purified PPi on the digital microfluidic platform. The standard curve was generated based on the enzymatic reaction with varying concentrations of PPi (stock concentrations of 20, 10, 5, 2.5, 1.25, 0.625, 0.3125, and 0 μM). A linear regression analysis was used to determine a coefficient of determination (R²) value of greater than 0.99. As shown in plot 1300, the standard curve exhibits good linear sensitivity to PPi concentrations of up to 2.5 μM. In all subsequent experiments, the standard curve was re-created for each cartridge run.

### 8.2 Pyrosizing of DNA Samples

For the DNA pyrosizing assay, the 1204 and 688 bp lambda-Hind III DNA fragments were used. The 1204 bp and 688 bp DNA fragments were amplified using a traditional bench thermocycle PCR instrument. The PCR products were gel purified on a 0.8% agarose gel. The DNA bands were excised and purified on a gel purification column (Invitrogen). Final elution time was about 5 minutes in a volume of 15 μL. Fragment purification was performed to ensure that no small DNA fragments (e.g., primer or other oligonucleotide) were present in the DNA sample.

The 20 μL ATPS/luciferase/luciferin enzyme mixture was prepared as described in Table 1. A 15 μL terminal deoxytransferase mix was prepared by diluting stock concentrations of 10× buffer, 2.5 mM CoCl₂, 10 mM ddATP, 200 μM APS, 1% Tween 20, and terminal deoxytransferase (20 U/μl) to final concentrations of 0.5 mM CoCl₂, 0.2 mM ddATP, 80 μM APS and 1.33 U/μL TdT. The DNA sample inputs were as follows: 1204 bp (533 ng/μL), 688 bp (355 ng/μL) and a 1:1 mix of 1204 bp (533 ng/μL) and 688 bp (355 ng/μL) samples. DNA samples (2.7 μL) were diluted with 1% Tween 20 to a final volume of 3 μL.

The digital microfluidic pyrosizing protocol included the following steps: The ATPS/luciferase/luciferin enzyme mixture (20 μL), TdT enzyme mix (15 μL) and each DNA fragment sample (3 μL) were loaded into separate reservoirs of a digital microfluidic cartridge and a run was initiated. For each PPi concentration, one droplet of a DNA fragment sample (˜100 nL) was dispensed and combined using droplet operations with one droplet of the TdT mix to yield a DNA/TdT droplet (˜200 nL). The DNA/TdT droplet was incubated for 30 min at 37° C. After the incubation period, the DNA/TdT droplet was split using droplet operations into two 100 nL DNA/TdT droplets. One 100 nL DNA/TdT droplet was combined using droplet operations with one droplet (˜100 nL) of the ATPS/luciferase/luciferin enzyme mix to generate the PPi chemiluminescent signal.

Two pyrosizing assays were performed using the 1204 bp purified samples. The PPi pyrosizing reaction predicted average fragment sizes of 1476 and 1127 bp, respectively. The 688 bp fragment produced too low of a signal and was not included in data analysis.

The data suggest that the PPi-based sizing approach using digital microfluidic technology is a feasible method for determining DNA fragment size. Samples must be purified of oligonucleotides, which because of their small size, produce extremely high amounts of PPi signal and therefore drown out signal derived from large fragments. For this application, any fragments above 1 kb are of interest, with fragments below this size considered too small to be of use during genome level analysis.

### Systems

The methods of the invention may be performed using droplet operations on a system of the invention. The droplet operations may be performed using a droplet actuator.

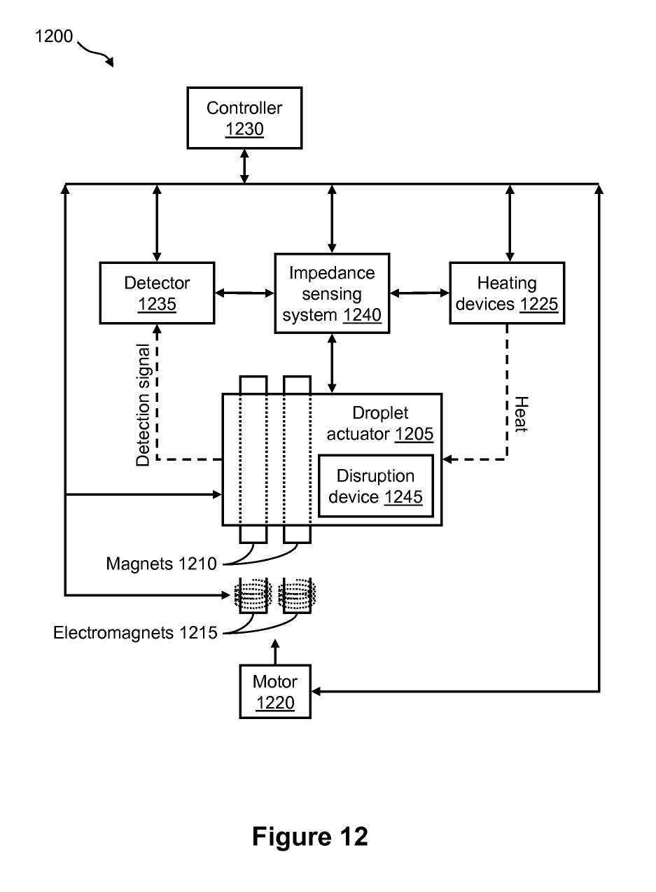

FIG. 12 illustrates a functional block diagram of an example of a microfluidics system 1200 that includes a droplet actuator 1205. Digital microfluidic technology conducts droplet operations on discrete droplets in a droplet actuator, such as droplet actuator 1205, by electrical control of their surface tension (electrowetting). The droplets may be sandwiched between two substrates of droplet actuator 1205, a bottom substrate and a top substrate separated by a droplet operations gap. The bottom substrate may include an arrangement of electrically addressable electrodes. The top substrate may include a reference electrode plane made, for example, from conductive ink or indium tin oxide (ITO). The bottom substrate and the top substrate may be coated with a hydrophobic material. Droplet operations are conducted in the droplet operations gap. The space around the droplets (i.e., the gap between bottom and top substrates) may be filled with an immiscible inert fluid, such as silicone oil, to prevent evaporation of the droplets and to facilitate their transport within the device. Other droplet operations may be effected by varying the patterns of voltage activation; examples include merging, splitting, mixing, and dispensing of droplets.

Droplet actuator 1205 may be designed to fit onto an instrument deck (not shown) of microfluidics system 1200. The instrument deck may hold droplet actuator 1205 and house other droplet actuator features, such as, but not limited to, one or more magnets and one or more heating devices. For example, the instrument deck may house one or more magnets 1210, which may be permanent magnets. Optionally, the instrument deck may house one or more electromagnets 1215. Magnets 1210 and/or electromagnets 1215 are positioned in relation to droplet actuator 1205 for immobilization of magnetically responsive beads. Optionally, the positions of magnets 1210 and/or electromagnets 1215 may be controlled by a motor 1220. Additionally, the instrument deck may house one or more heating devices 1225 for controlling the temperature within, for example, certain reaction and/or washing zones of droplet actuator 1205. In one example, heating devices 1225 may be heater bars that are positioned in relation to droplet actuator 1205 for providing thermal control thereof.

A controller 1230 of microfluidics system 1200 is electrically coupled to various hardware components of the invention, such as droplet actuator 1205, electromagnets 1215, motor 1220, and heating devices 1225, as well as to a detector 1235, an impedance sensing system 1240, and any other input and/or output devices (not shown). Controller 1230 controls the overall operation of microfluidics system 1200. Controller 1230 may, for example, be a general purpose computer, special purpose computer, personal computer, or other programmable data processing apparatus. Controller 1230 serves to provide processing capabilities, such as storing, interpreting, and/or executing software instructions, as well as controlling the overall operation of the system. Controller 1230 may be configured and programmed to control data and/or power aspects of these devices. For example, in one aspect, with respect to droplet actuator 1205, controller 1230 controls droplet manipulation by activating/deactivating electrodes.

In one example, detector 1235 may be an imaging system that is positioned in relation to droplet actuator 1205. In one example, the imaging system may include one or more light-emitting diodes (LEDs) (i.e., an illumination source) and a digital image capture device, such as a charge-coupled device (CCD) camera.

Impedance sensing system 1240 may be any circuitry for detecting impedance at a specific electrode of droplet actuator 1205. In one example, impedance sensing system 1240 may be an impedance spectrometer. Impedance sensing system 1240 may be used to monitor the capacitive loading of any electrode, such as any droplet operations electrode, with or without a droplet thereon. For examples of suitable capacitance detection techniques, see Sturmer et al., U.S. Patent Application Publication No. US20100194408, entitled “Capacitance Detection in a Droplet Actuator,” published on Aug. 5, 2010; and Kale et al., U.S. Patent Application Publication No. US20030080143, entitled “System and Method for Dispensing Liquids,” published on May 1, 2003; the entire disclosures of which are incorporated herein by reference.

Droplet actuator 1205 may include disruption device 1245. Disruption device 1245 may include any device that promotes disruption (lysis) of materials, such as tissues, cells and spores in a droplet actuator. Disruption device 1245 may, for example, be a sonication mechanism, a heating mechanism, a mechanical shearing mechanism, a bead beating mechanism, physical features incorporated into the droplet actuator 1205, an electric field generating mechanism, a thermal cycling mechanism, and any combinations thereof. Disruption device 1245 may be controlled by controller 1230.

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network may even include wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

### CONCLUDING REMARKS

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

1-97. (canceled)

98. A method of determining the average size of DNA fragments in a DNA sample, the method comprising:
a. conducting a pyrosequencing reaction comprising combining the DNA sample and pyrosequencing reagents, wherein the pyrosequencing reaction is conducted without determining the nucleic acid sequences of the DNA fragments in the DNA sample, whereby the pyrosequencing reaction yields a detectable pyrophosphate concentration;
b. determining the pyrophosphate concentration; and
c. determining the average size of DNA fragments in the DNA sample based on the pyrophosphate concentration.

99. The method of 98, wherein combining the DNA sample and pyrosequencing reagents comprises incubating the DNA sample with terminal deoxytransferase and ddATP, wherein dideoxynucleotides are incorporated into the DNA fragments in the DNA sample.

100. The method of claim 98, wherein pyrophosphate concentration in the DNA sample is determined in moles/liter.

101. The method of claim 100, wherein determining the average size of DNA fragments in the DNA sample based on the pyrophosphate concentration comprises:
i. determining a DNA concentration in the DNA sample in grams/liter;
ii. calculating the average molecular weight of the DNA fragments in the DNA sample in grams/mole, comprising dividing the DNA concentration in grams/liter by ½ the pyrophosphate concentration in moles/liter; and
iii. calculating the average size of DNA fragments in the DNA sample, comprising dividing the average molecular weight of DNA in grams/mole by 660 grams/base pair.

102. The method of claim 98, wherein determining the pyrophosphate concentration comprises performing a chemiluminescence assay on the DNA sample.

103. The method of claim 98, wherein determining a DNA concentration in the DNA sample comprises performing qPCR on the DNA sample.

104. The method of claim 98, wherein the average size of DNA fragments in the DNA sample is determined for diagnosing or screening for a nucleotide repeat disorder.

105. The method of claim 104, wherein the nucleotide repeat disorder is a polyglutamine disease.

106. The method of claim 105, wherein the polyglutamine disease is selected from the group consisting of dentatorubropallidoluysian atrophy, Huntington's disease, spinobulbar muscular atrophy, and spinocerebellar ataxia types 1, 2, 3, 6 and 7.

107. The method of claim 104, wherein the nucleotide repeat disorder is a non-polyglutamine disease.

108. The method of claim 107, wherein the non-polyglutamine disease is selected from the group consisting of fragile X Syndrome, fragile XE mental retardation, Friedreich's Ataxia, myotonic dystrophy, and spinocerebellar ataxia types 8 and 12.

109. The method of claim 98, wherein the DNA sample comprises a biological sample.

110. The method of claim 109, wherein the biological sample is collected from a subject.

111. The method of claim 110, wherein the biological sample comprises a cheek swab.

112. The method of claim 110, wherein the biological sample comprises a dried blood spot.

113. The method of claim 110, wherein the subject is suspected of having a nucleotide repeat disorder.

114. The method of claim 113, wherein the nucleotide repeat disorder is a polyglutamine disease.

115. The method of claim 114, wherein the polyglutamine disease is selected from the group consisting of dentatorubropallidoluysian atrophy, Huntington's disease, spinobulbar muscular atrophy, and spinocerebellar ataxia types 1, 2, 3, 6 and 7.

116. The method of claim 113, wherein the nucleotide repeat disorder is a non-polyglutamine disease.

117. The method of claim 116, wherein the non-polyglutamine disease is selected from the group consisting of fragile X Syndrome, fragile XE mental retardation, Friedreich's Ataxia, myotonic dystrophy, and spinocerebellar ataxia types 8 and 12.

118. A method of determining the average size of DNA fragments in a DNA sample, the method comprising:
a. conducting a pyrosequencing reaction comprising combining the DNA sample and pyrosequencing reagents, wherein the pyrosequencing reaction is conducted without determining the nucleic acid sequences of the DNA fragments in the DNA sample, whereby the pyrosequencing reaction yields a detectable pyrophosphate concentration;
b. determining the pyrophosphate concentration, wherein pyrophosphate concentration in the DNA sample is determined in moles/liter; and
c. determining the average size of DNA fragments in the DNA sample based on the pyrophosphate concentration, comprising:
i. determining a DNA concentration in the DNA sample in grams/liter;
ii. calculating the average molecular weight of the DNA fragments in the DNA sample in grams/mole, comprising dividing the DNA concentration in grams/liter by ½ the pyrophosphate concentration in moles/liter; and
iii. calculating the average size of DNA fragments in the DNA sample, comprising dividing the average molecular weight of DNA in grams/mole by 660 grams/base pair.
